(19) United States
(12) Patent Application Publication
(10) Pub. No.: US 2026/0297193 A1
(43) Pub. Date: Oct. 1, 2026

(54) TCR-MIMIC BISPECIFIC ANTIBODIES FOR HIV-1

(71) Applicant: THE JOHNS HOPKINS UNIVERSITY, Baltimore (MD)

(72) Inventors: Srona A. Sengupta, Baltimore, MD (US); Robert Siliciano, Chevy Chase, MD (US); Scheherazade Sadegh-nasseri, Baltimore, MD (US); Jacqueline Douglass, Baltimore, MD (US)

(21) Appl. No.: 19/473,394

(22) PCT Filed: Apr. 7, 2023
(86) PCT No.: PCT/US2023/017904

§ 371 Date: Oct. 7, 2025

(51) Int. Cl. C07K 16/28; A61P 37/04; C07K 16/114; G01N 33/569

(52) U.S. Cl. CPC C07K 16/2809; A61P 37/04; C07K 16/114; G01N 33/56988; C07K 2317/14; C07K 2317/31; C07K 2317/622; C07K 2317/626; G01N 2333/16

(57) ABSTRACT

T cell receptor (TCR)-mimic antibodies are constructed having specificity for HIV-peptide-MHC complexes (pMHC). Single-chain diabody forms of these antibodies recognize distinct epitopes in Gag and RT in a specific manner and induce T-cell-mediated killing of HIV infected CD4⁺ T cells. These pMHC-based immunotherapeutic approaches also result in the elimination of the latent reservoir and aid in identifying effective latency reversing agents.

### BACKGROUND

HIV-1 is incurable due to a long-lived reservoir in resting CD4⁺T cells that harbor latent, replication-competent proviruses (1-5). When resting CD4⁺ T cells are activated by antigen or other stimuli, the transcriptional environment becomes permissive for viral gene expression, and infectious virions can be released. If cART is interrupted, exponential viral replication ensues (6, 7), eventually leading to acquired immune deficiency (AIDS). Due to the extremely slow decay of the latent reservoir (t½~3.6 years), persons living with HIV-1 (PLWH) must remain on cART for life (8-11).

Efforts to cure HIV-1 infection have focused on the “shock and kill” strategy which relies upon latency-reversing agents (LRAs) to induce viral gene expression, revealing these infected cells to the immune system (“shock”). These cells can then be targeted for cytolysis by CD8⁺ cytolytic T lymphocytes (CTL) or NK cells (“kill”) (12). CTL-mediated killing requires T cell receptor (TCR) recognition of short (8-11 amino acid) peptide fragments of HIV-1 proteins presented on major histocompatibility class I (MHC-I) molecules (13). In principle, “shock and kill” should reduce the reservoir. Yet, while certain LRAs have caused viral “blips” (transient increases plasma virus into the detectable range) in PLWH on cART, current LRAs alone have not reduced the latent reservoir (14-17). One explanation is compromised CTL function not fully restored by cART (18, 19). Recent studies have shown that pre-stimulation of CTLs is required for elimination of infected cells following latency reversal (20). Additionally, the latent reservoir of PLWH contains proviruses with escape mutations in dominant CTL epitopes (21), and certain LRAs impair CTL function (22-25). Therefore, curative approaches will likely require therapeutic agents that promote killing of infected cells in combination with effective LRAs.

### SUMMARY

T cell receptor (TCR)-mimic antibodies to HIV-1 peptide-MHC complexes (pMHC) are provided. Epitopes in Gag and reverse transcriptase (RT) were identified and quantified. Sequences that bound these pMHC were cloned into a single-chain diabody backbone (scDb) sequence, such that one fragment is specific for an HIV-1 pMHC and the other fragment binds to CD3ε, essential signal transduction subunit of the TCR.

In certain aspects, a T cell receptor (TCR)-mimic (TCRm) antibody comprises two binding domains, wherein (i) a first binding domain specifically binds to a retroviral peptide complexed with a major histocompatibility complex molecule (pMHC) and (ii) a second binding domain specifically binds to T cell signal transduction peptide. In certain embodiments, the retrovirus comprises human immunodeficiency virus (HIV), human T-lymphotropic virus type 1 (HTLV-1) or human T-lymphotropic virus type 2 (HTLV-II). In certain embodiments, the retroviral peptide comprises HIV peptides, HTLV-1 or HTLV-2 peptides. In certain embodiments, the retroviral peptide is an HIV peptide. In certain embodiments, the HIV peptide is an HIV-1 peptide or an HIV-2 peptide. In certain embodiments, the HIV-1 peptide comprises Gag, Pol, Env, or Reverse Transcriptase (RT). In certain embodiments, the major histocompatibility complex (MHC) MHC-I, MHC-II or human leukocyte antigen E (HLA-E). In certain embodiments, the pMHC comprises an HIV-1, Gag, Pol, Env, or Reverse Transcriptase (RT) peptide complexed to MHC-I or HLA-E. In certain embodiments, the T cell signal transduction peptide comprises a CD3 peptide. In certain embodiments, the CD3 peptide comprises CD3γ, CD3δ, CD3ε, or CD3ζ. In certain embodiments, the CD3 peptide is CD3ε. In certain embodiments, the second antigen binding domain specifically binds to a CD3ε epitope. In certain embodiments, the antigen binding domains comprise: polyclonal antibodies or fragments thereof, monoclonal antibodies or fragments thereof, antigen-binding antibody fragments, (Fab) fragments, F(ab′)2 fragments, Fab′ fragments, Fv fragments, recombinant IgG (rIgG) fragments, variable heavy chain (VH) regions, single chain antibody fragments, single chain variable fragments (scFv), single domain antibodies, bispecific, antibodies, diabodies, single-chain diabodies (scDb), triabodies, and tetrabodies, tandem di-scFv, or tandem tri-scFvs. In certain embodiments, the antigen binding domains comprise single-chain diabodies (scDb).

In another aspect, a method of treating a subject diagnosed with a human immunodeficiency virus (HIV) infection, comprises administering to the subject a therapeutically effective amount of a bispecific T cell receptor (TCR)-mimic (TCRm) antibody comprising two binding domains, wherein (i) a first binding domain specifically binds to an HIV peptide complexed with a major histocompatibility complex molecule (pMHC) and (ii) a second binding domain which specifically binds to T cell signal transduction peptide. In certain embodiments, the HIV peptide is an HIV-1 peptide or an HIV-2 peptide. In certain embodiments, the HIV-1 peptide comprises Gag, Pol, Env, or Reverse Transcriptase (RT). In certain embodiments, the major histocompatibility complex (MHC) comprises MHC-I, MHC-II or human leukocyte antigen E (HLA-E). In certain embodiments, the pMHC comprises an HIV-1, Gag, Pol, Env, or Reverse Transcriptase (RT) peptide complexed to MHC-I or HLA-E. In certain embodiments, the T cell signal transduction peptide comprises a CD3 peptide. In certain embodiments, the CD3 peptide comprises CD3γ, CD3δ, CD3ε, or CD3ζ. In certain embodiments, the CD3 peptide is CD3ε. In certain embodiments, the antigen binding domains comprise single-chain diabodies (scDb). In certain embodiments, the method further comprises administering one or more secondary therapeutics. In certain embodiments, the secondary therapeutics comprise anti-viral agents, gene-editing agents, latency reversing agents (LRAs) or combinations thereof.

In another aspect, a method of producing a bispecific diabodies comprises obtaining a biological sample from a subject infected with a virus or infecting a cell in vitro with a virus, eluting peptides comprising MHC and virus peptides from the biological sample or in vitro infected cells, utilizing predictive algorithms combined with chromatography and mass spectrometry to identify high-affinity cytotoxic T cell (CTL) epitopes which bind to major histocompatibility complex (MHC) alleles, measuring fragmentation patterns of and elution positions relative to a set of retention time (RT) peptides, synthesizing identified viral peptides complexed with MHC molecules (pMHC), screening a phage library to identify single chain variable fragments (scFv) which specifically bind to viral peptides complexed with MHC molecules (pMHC), converting the scFv fragments to diabodies, and producing bispecific diabodies. In certain embodiments, the prediction of peptides comprises a Poisson detection liquid chromatography data independent acquisition mass spectrometry (LC-DIAMS). In certain embodiments, the LC-DIAMS comprises in silico algorithms to predict putative high-affinity CTL epitopes binding to MHC alleles of interest. In certain embodiments, the biological sample from the subject comprises infected cells and immune cells. In certain embodiments, the method further comprises activating the immune cells in vitro.

In another aspect, a method of identifying latency reversing agents (LRAs), comprises producing bispecific diabodies embodied herein, which specifically bind to human immunodeficiency virus (HIV) peptides complexed with MHC molecules (pMHC), screening HIV infected cells against a library of candidate agents; contacting the HIV infected cells with the pMHC specific diabodies; assaying for changes in expression or levels of pMHC antigens; thereby identifying latency reversing agents. In certain embodiments, the screening assay is a high-throughput screening assay.

In another aspect, the pMHCs comprise one or more mutations.

In another aspect, a bispecific single chain diabody (scDb) comprises SEQ ID NOs: 108 and 109. In certain embodiments, the scDb a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 108 or 109. In certain embodiments, the scDb comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 108 or 109. In certain embodiments, the scDb comprises at least an 80%, 85%, 86%, 87%, 88%, 89%, 90%, 91%, 92%, 93%, 94%, 95%, 96%, 97%, 98%, or 99%, sequence identity to SEQ ID NO: 108 or 109. In certain embodiments, the scDb comprises at least a 90%, 91%, 92%, 93%, 94%, 95%, 96%, 97%, 98%, or 99%, sequence identity to SEQ ID NO: 108 or 109.

In another aspect, a bispecific single chain diabody (scDb) comprises SEQ ID NOs: 110 and 111. In certain embodiments, the scDb a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 110 or 111. In certain embodiments, the scDb comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 110 or 111. In certain embodiments, the scDb comprises at least an 80%, 85%, 86%, 87%, 88%, 89%, 90%, 91%, 92%, 93%, 94%, 95%, 96%, 97%, 98%, or 99%, sequence identity to SEQ ID NO: 110 or 111. In certain embodiments, the scDb comprises at least a 90%, 91%, 92%, 93%, 94%, 95%, 96%, 97%, 98%, or 99%, sequence identity to SEQ ID NO: 110 or 111.

In another aspect, a bispecific single chain diabody (scDb) comprises SEQ ID NOs: 112 and 113. In certain embodiments, the scDb a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 112 and 113. In certain embodiments, the scDb comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 112 and 113. In certain embodiments, the scDb comprises at least an 80%, 85%, 86%, 87%, 88%, 89%, 90%, 91%, 92%, 93%, 94%, 95%, 96%, 97%, 98%, or 99%, sequence identity to SEQ ID NO: 112 and 113. In certain embodiments, the scDb comprises at least a 90%, 91%, 92%, 93%, 94%, 95%, 96%, 97%, 98%, or 99%, sequence identity to SEQ ID NO: 112 and 113.

In another aspect, a bispecific single chain diabody (scDb) comprises SEQ ID NOs: 114 and 115. In another aspect, a bispecific single chain diabody (scDb) comprises SEQ ID NOs: 114 and 115. In certain embodiments, the scDb a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 114 and 115. In certain embodiments, the scDb comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 114 and 115. In certain embodiments, the scDb comprises at least an 80%, 85%, 86%, 87%, 88%, 89%, 90%, 91%, 92%, 93%, 94%, 95%, 96%, 97%, 98%, or 99%, sequence identity to SEQ ID NO: 114 and 115. In certain embodiments, the scDb comprises at least a 90%, 91%, 92%, 93%, 94%, 95%, 96%, 97%, 98%, or 99%, sequence identity to SEQ ID NO: 114 and 115.

In another aspect, a T cell receptor (TCR)-mimic (TCRm) antibody comprising two binding domains, wherein (i) a first binding domain specifically binds to an HIV peptide complexed with a major histocompatibility complex molecule I (pMHC) and (ii) a second binding domain which specifically binds to a CD3ε epitope.

In another aspect, the scDbs, comprise one or more mutations which increase specific binding as compared to the unmodified scDb. In certain embodiments, the scDbs are modified to increase or decrease in vivo half-life.

In certain embodiments, the antigen binding domain is or comprises an antibody or antibody fragment, aptamers, proteins and the like. In certain embodiments, the antibodies are human antibodies, including any known to bind a targeting molecule. The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, single-chain diabodies (scDb), triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In another aspect, a method of activating an immune response to human immunodeficiency virus (HIV), comprises administering to a subject diagnosed with an HIV infection, a therapeutically effective amount of a T cell receptor (TCR)-mimic (TCRm) antibody comprising two binding domains, wherein (i) a first binding domain specifically binds to an HIV peptide complexed with a major histocompatibility complex molecule (pMHC) and (ii) a second binding domain which specifically binds to T cell signal transduction peptide or bispecific single chain diabody (scDb) comprising SEQ ID NOs: 108-115. In certain embodiments, the HIV infection is latent. In certain embodiments, further comprises administering one or more latency reversing agents (LRAs). In certain embodiments, further comprises administering one or more anti-viral agents.

Preferred systems are disclosed in Srona Sengupta et al., (2022). PNAS 119 (15) e2123406119, doi.org/10.1073/pnas.2123406119, incorporated herein by reference in its entirety.

### Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, within 5-fold, and also within 2-fold, of a value. Where particular values are described in the application and claims, unless otherwise stated the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C”, “one or more of A, B, and C” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the term “affinity” is meant as a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the disclosure at any stage during clinical trials, during pre-trial testing, or following FDA-approval.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, gene-editing agents, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

By “ameliorate” is meant decrease, suppress, attenuate, diminish, arrest, or stabilize the development or progression of a disease.

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The amino acids can be non-naturally occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (0β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “antibody” includes monoclonal antibodies (including full length antibodies which have an immunoglobulin Fc region), antibody compositions with polyepitopic specificity, multispecific antibodies (e.g., bispecific antibodies, diabodies, single-chain diabodies (scDbs) and single-chain molecules, as well as antibody fragments (e.g., Fab, F(ab′)₂, Fab′, and Fv). The term “immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H1}). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2. The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H1}). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H1} domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “bispecific” as provided herein is used according to its conventional meaning well known in the art and refers to a bispecific recombinant protein capable of simultaneously binding to two different antigens. In contrast to traditional monoclonal antibodies, bispecific antibodies consist of two independently different antibody regions (e.g., two single-chain variable fragments (scFv)), each of which binds a different antigen.

As used herein, the transitional term “comprising,” which is synonymous with “including,” “containing,” or “characterized by,” is inclusive or open-ended and does not exclude additional, unrecited elements or method steps. When used herein the term “comprising” can be substituted with the term “containing” or “including” or sometimes when used herein with the term “having.” By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

The term “diabodies” refers to small antibody fragments prepared by constructing sFv fragments with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993). A single-chain Diabody (scDb) is a derivative of diabody. In diabodies, two polypeptide chains are assembled noncovalently. scDb connects the first chain and the second chain of diabodies by a flexible linker, transforming the heterodimeric format of diabody into a single-chain format. In scDb, all four variable domains are on one chain and assemble into diabody-like molecule. The middle linker can force correct assembly and improve stability. scDb can also be fused with Fc or CH₃ domain to exert Fc-mediated functions and/or extend half-life.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate.

An “epitope” is a term well understood in the art and means any chemical moiety that exhibits specific binding to an antibody or T cell receptor. An “antigen” is a moiety or molecule that contains an epitope, and, as such, also specifically binds to antibody or T cell receptor.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) can recognize and bind antigen, although at a lower affinity than the entire binding site.

The term “hinge” or “hinge region” refers to a flexible connector region, e.g. natural or synthetic polypeptides, or any other type of molecule, providing structural flexibility and spacing to flanking polypeptide regions.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit, or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures. The residues from each of these HVRs are noted below.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises a naive CD4⁺ T cell. In some aspects, an immune cell comprises a memory CD4⁺ T cell. In some aspects, an immune cell comprises an effector CD4⁺ T cell. In some aspects, an immune cell comprises a naïve CD8⁺ T cell. In some aspects, an immune cell comprises a memory CD8⁺ T cell. In some aspects, an immune cell comprises an effector CD8⁺ T cell. In some aspects, an immune cell comprises a gamma delta T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

The term “latency”, as used herein is a reversible state of non-productive infection. Latent proviruses are those that could theoretically be induced to express LTR-driven transcripts, but at a given time do not initiate or maintain gene expression due to transcriptional or epigenetic constraints (e.g. lack of key transcription factors and co-regulators or suppressive chromatin modifications).

The term “latency reactivating agent”, as used herein refers to the ability of a particular agent to reactivate a latent virus, e.g. HIV in infected cells to replicate and produce virions.

The term “linker”, also referred to as a “spacer” or “spacer domain” as used herein, refers to a chemical linker, or an amino acid or sequence of amino acids that that is optionally located between two amino acid sequences in the diabodies embodied herein.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, monoclonal antibodies may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used in this specification and the appended claims, the term “or” is generally employed in its sense including “and/or” unless the content clearly dictates otherwise and should be understood to mean “either or both” of the elements so conjoined, e.g., elements that are conjunctively present in some cases and disjunctively present in other cases.

“Parenteral” administration of an immunogenic composition includes, e.g., subcutaneous (s.c.), intravenous (i.v.), intramuscular (i.m.), intravitreal (i.v.i.), intra-cisterna magna (i.c.m.), or intrasternal injection, or infusion techniques.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994). “Functional fragments” of the antibodies of the disclosure comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

As used herein, “specifically binding” or “binds specifically to” refers to an antibody or fragments thereof, e.g. scFvs, scDb's, which selectively or preferentially bind to an antigen. The binding affinity is generally determined using a standard assay, such as Scatchard analysis, or surface plasmon resonance technique (e.g. using BIACORE®).

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof, an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the helicases described herein, one or more amino acids of the helicase are substituted with one or more unnatural amino acids and/or one or more natural amino acids.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

### BRIEF DESCRIPTION OF THE DRAWINGS

FIGS. 1A-1E demonstrate the physical identification of HLA-A*02:01-bound HIV-1 peptides by mass spectrometry from infected CD4⁺T cells. FIG. 1A: Method for generating infected CD4⁺T cells for physical detection of HLA-A*02:01-bound HIV-1 epitopes. FIGS. 1B-1D: Detection of the native and isotope-labeled forms of HIV-1 peptides ALTEVVPLT (ALT), VLAEAMSQV (VLA), and ILKEPVHGV (ILK) in GFP+ cells by targeted LC-MS/MS. Detection is indicated by elution coincidence between the extracted ion chromatogram (XIC) for the peptide's precursor m/z (top traces, in black) and the Poisson chromatogram, related to the probability of the peptide's fragmentation pattern (bottom, inverted blue trace, scaled to XIC max). The heavy and light forms of the peptides must also coelute, being distinguished only by different precursor masses. The relative XIC peak amplitudes, scaled by the amount of added heavy peptide indicate the amount of the native peptide on the GFP+ cells. This can be converted into copies per cell (FIGS. 7A, 7B, 8A-8D, 9A-9C). FIG. 1E: Three HLA-A*02:01 epitopes of HIV-1 are identified at different copy numbers per cell via LC-DIAMS and were the targets for generating specific single-chain diabodies (scDbs) (see FIGS. 2A-2E). FIG. 1F: RT epitopes are highlighted on the PBD structure 5TXM.

FIGS. 2A-2E demonstrate the isolation of HIV-1 pMHC-specific scFv and conversion to a single-chain diabody format. FIG. 2A: Schematic of phage panning and characterization for the isolation of phage clones bearing single-chain variable fragments (scFvs) specific for HIV pMHC-I. V_{L} and V_{H} sequences from the scFv of specific phage clones are cloned and expressed as single-chain diabodies (scDbs). Secreted scDbs are purified by nickel chromatography and tested for functionality in co-cultures with target cells (peptide-pulsed cells or infected CD4⁺T cells) and pre-stimulated CD8-T cells. FIG. 2B: Monoclonal phage clones found to bind specifically to target pMHC-I by ELISA (FIGS. 10A-10C) were concentrated and tested for binding to T2 cells pulsed with relevant or irrelevant peptides. Phage specific for ALT pMHC-I (i.e. HA phage clones) bind to ALT-pulsed T2 cells (green) but not ILK-pulsed (orange) or VLA-pulsed (purple) cells. Specific binding of HI and HV monoclonal phage are also shown. FIG. 2C: scFv from specific phage clones from FIG. 2B and FIG. 11D were cloned and expressed as scDbs against ALT (HA-scDb), ILK (HI-scDb), and VLA (HV-scDb). scDbs were screened for specific activation of CD8⁺T cells as measured by surface CD107 when co-cultured with T2 cells pulsed with relevant or irrelevant peptides. Boxes indicate the HA, HI, and HV scDb exhibiting the greatest potency and specificity of CD107 activation. Data shown represent mean±range of two biological replicates. Increasing concentrations of FIG. 2D: HA29-scDb or FIG. 2E: HI12-scDb binding to 1 μg/ml immobilized ALT/HLA-A*02:01 (green) or ILK/HLA-A*02:01 (orange), respectively, was assessed via ELISA. Data shown represent mean±SD of three technical replicates.

FIGS. 3A-3B demonstrate that HA29 and HI12-scDbs induce polyfunctional CD8⁺ T cell responses. T2 cells were pulsed with the ALT or ILK peptides at the indicated concentrations. 10×10⁴ peptide-pulsed cells were co-cultured with 5×10⁴ pre-activated CD8⁺T cells (1:2 E to T) in the presence of 0.25 nM HA29 or HI12 scDbs for 72 hrs. Supernatants were assayed with Legendplex or MIP1β ELISAs. Specific activation of CD8⁺T cells by HA29 or HI12 was observed by the secretion of Granzyme A and IFN-γ. in response to cells pulsed with the cognate but not irrelevant peptide. Data indicate mean±range of two biological replicates. Similar specific responses were also observed using assays for other effector molecules (FIGS. 14A, 14B).

FIGS. 4A-4F demonstrate that HIV-specific scDbs have high affinity for their cognate pMHC-I. FIG. 4A: HA29-scDb binding to ALT/HLA-A*02:01 was measured with multicycle kinetics using SPR at concentrations up to 400 nM. FIGS. 4B-4C: No binding was observed with HA29 against irrelevant epitopes ILK/HLA-A*02:01 and VLA/HLA-A*02:01 at all concentrations tested. FIG. 4D: HI12-scDb binding to ILK/HLA-A*02:01 was measured with multicycle kinetics using SPR. No binding was observed with ILK against irrelevant epitopes ALT/HLA-A*02:01 and VLA/HLA-A*02:01 at all concentrations tested. All SPR measurements were done in duplicate. FIGS. 4E-4F: No binding was observed with ILK against irrelevant epitopes ALT/HLA-A*02:01 and VLA/HLA-A*02:01 at all concentrations tested. All SPR measurements were done in duplicate.

FIGS. 5A-5D demonstrate that HA29 and HI-12 scDbs induce killing of target cells bearing relevant pMHC-I. T2 cells were pulsed with ALT or ILK peptides and β2M and stained with CFSE or CTV, respectively. 15×10⁴ pre-activated CD8⁺T cells were cultured with 5×10⁴ peptide-pulsed, CFSE- and CTV-stained cells (3:1:1 E:T) in the presence of HA29 or H12 scDbs for 18 hrs and assayed for specific killing. Representative flow-cytometry plots showing the frequency of the ALT-pulsed (CFSE^{hi}) and ILK-pulsed (CTV^{hi}) populations in co-cultures with FIG. 5A: no scDb, 250 pM of isotype H2-scDb, and 250 pM of pan-A2-scDb; FIG. 5B: increasing doses of the HA29-scDb; or FIG. 5C: increasing doses of HI12-scDb. Flow plots were gated on lymphocyte size, single cells, and viable cells. Numbers by the gates indicate bead-corrected cell counts. FIG. 5D: Percent reduction of ALT-pulsed (CFSE^{hi}) or ILK-pulsed (CTV^{hi}) populations mediated by HA29- or HI12-scDbs. Data represent the mean±SD of four biological replicates.

FIGS. 6A-6E demonstrate that HA29-scDb induces dose-dependent viral suppression. FIG. 6A: Representative flow plots from a suppression assay showing the dramatic decrease in viable GFP⁺ cells with increasing concentrations of HA29-scDb compared to co-cultures containing isotype (H2) scDb. Activated CD4⁻ T cells from an A2-expressing healthy donor were infected with ΔEnv-NL4.3-EGFP and co-cultured with autologous pre-stimulated CD8⁺T cells for 72 hrs before flow cytometric analysis (see Methods). FIG. 6B: % of live GFP⁺ cells remaining after 72 hours of co-culture with HA29, HI12, or H2 scDbs and autologous CD8⁺ T cells. Results are expressed as a % of the live GFP⁺ cells remaining in cultures without scDbs. FIG. 6C: Suppression assays using cells from three A2⁺ and one A2⁻ donor. Results are expressed as % of the live GFP⁺ cells remaining relative to cultures without scDbs. FIG. 6D: Suppression of a replication-competent reservoir isolate 33A10 (56). Healthy donor CD4⁺T cells were activated, infected with 33A10, and co-cultured with autologous CD8⁺T cells in in the presence of 0.25 nM of the HA29, HI12, or H2 scDbs. Supernatant p24 was measured by ELISA after 1, 3, and 5 days of co-culture. Reduction in p24 was normalized to the no scDb control. FIG. 6E: Target cells (infected cells or infected cells pulsed with 10 μg/ml ILK peptide) were co-cultured with autologous CD8⁺T cells in the presence of the HI12 or H2 scDbs. Results are expressed as a % of the live GFP⁺ cells remaining in cultures without scDbs. Data in represent the mean±SD of three independent experiments analyzed by two-way ANOVA followed by Tukey's Multiple Comparison Test. Data in FIG. 6C represent the mean±SD of four independent experiments analyzed by two-way ANOVA followed by Tukey's Multiple Comparison Test. Data in FIGS. 6D and 6E represent the mean and range of two technical replicates and were analyzed by two-way ANOVA followed by Tukey's Multiple comparison test. *p<0.05, **p<0.01, ***p<0.001, ****p<^{0.0001.}

FIGS. 7A-7B demonstrate the Poisson LC-DIAMS detection of HLA-A*02:01-bound ALT and VLA peptides from 2.5×10⁶ GFP⁺ CD4⁺T cells. FIGS. 7A, 7B: Detection of ALTEVVPLT (ALT) and VLAEAMSQV (VLA) HIV-1 peptides in GFP⁺ cells, indicated by the presence of coeluting peaks in the precursor XIC (top black trace) and the Poisson chromatogram (bottom, inverted blue trace). The elution position of the coincident peaks must also be consistent with the elution mapping as determined by an LC-DIAMS run of the synthetic set containing a set of mapping peptides shared in both the synthetic and sample runs (see Methods). Of the 64 HIV-1 HLA-A*02:01-binding peptides assayed, these two were the only ones detected in the +2-charge state. None of the 64 were detected in GFP⁻ cells.

FIGS. 8A-8D demonstrate the quantitation of HLA-A*02:01-bound peptides ALT and VLA detected from 2.5e6 GFP⁺ CD4⁺T cell by targeted LC-MS/MS. 100 attomoles of isotope-labeled heavy ALT and VLA peptides were added. FIG. 8A: Poisson detection plot shows heavy ALT, hence light ALT, eluting at scan 767. FIG. 8B: The mass spectrum at scan 767 shows precursor ion amplitudes for both light (30,000) and heavy (13,300) forms. 30000/13300*100=226 attomoles. Over 2.5 million cells this is a copy number of 54 per cell. FIGS. 8C, 8D: As above with 2500/5850*100=43 attomoles or 10 copies/cell.

FIGS. 9A-9C demonstrate the quantitation of HLA-A*02:01-bound ILK detected from 2.5e6 GFP⁺ CD4⁺T cell by targeted LC-MS/MS. 200 attomoles of isotope-labeled heavy ILK were added. FIG. 9A: Poisson detection plot shows heavy ILK, hence light ILK, eluting at scan 241. FIG. 9B: The mass spectrum at scan 241 shows the precursor ion amplitude of the heavy form but to see the light form light form requires an expanded scale (FIG. 9C). That this minor peak is light ILK is shown by its Poisson signature in FIGS. 1A-1E. The base isotope peak at 331.203 is overlapping with background ions, hence the first isotope peaks (marked with asterisks) is used for quantitation. 570/22400*200=5 attomoles which is less than 1 copy/cell.

FIGS. 10A-10C demonstrate the selection of scFv specific for ALT, ILK, and VLA pMHC. Monoclonal phage were amplified in bacteria in a 96-well plate format after 4 rounds of selection and then incubated in streptavidin ELISA plates coated with biotinylated ALT pHLA-A2 (FIG. 10A), ILK pHLA-A2 (FIG. 10B), VLA pHLA-A2 (FIG. 10C), or an isotype pHLA-A2 control. Plates were washed and phage were detected by absorbance at 450 nm using rabbit anti-M13 and an HRP-conjugated mouse anti-rabbit secondary antibody. Arrows indicate phage whose scFv sequences were utilized to construct the HA29, HI12, HV114, and HV115 scDbs described in the manuscript. Well H12 was not inoculated with phage and therefore served as a no phage control.

FIGS. 11A-11G demonstrate the flow cytometry characterization of specific monoclonal phage. pMHC-specific monoclonal phage clones that bound >2 fold higher to the target vs irrelevant pMHC-A2 in monoclonal ELISAs were tested for binding on T2 (HLA-A*02:01⁺) cells pulsed with 50 μg/ml of the indicated peptide. Phage binding was assessed by flow cytometry using a rabbit anti-M13 primary antibody and a donkey anti-Rabbit secondary antibody conjugated to phycoerythrin (PE). Phage binding was quantified by PE median fluorescence intensity (MFI), and the ratio of monoclonal phage bound to cells pulsed with the cognate or irrelevant peptides plus beta-2 microglobulin (B2M) was used to rank pMHC-specific phage by specificity. FIGS. 11A-11C: The MFI ratio of HA, HI, or HV-specific phage binding to cognate (ALT, ILK, or VLA peptides, respectively) versus irrelevant peptide-pulsed cells is plotted. FIG. 11D: Peptide stabilization of cell-surface HLA-A2 on T2 cells is shown with all three candidate target peptides. FIG. 11E: Representative histograms of HA29 phage clone binding to T2 cells pulsed with 10 μg/ml B2M only (gray) or B2M+50 μg/ml ALT (green), ILK (orange), or VLA peptides. A large shift in phage binding with ALT-pulsed cells is observed. FIG. 11F: Monoclonal phage clone HI12 shows increased binding to T2 cells pulsed with the cognate peptide (ILK) versus irrelevant peptides (VLA and ALT). FIG. 11G: Additional monoclonal phage screening via flow cytometry for VLA-specific phage yielding specific clones HV115 and HV154 are shown.

FIGS. 12A-12D demonstrate a selection of HA29 and HI12-scDbs for further characterization. FIG. 12A: T2 cells were pulsed with 1 ug/ml ALT or ILK peptides and 10 ug/ml B2M and co-cultured with pre-activated CD8⁺T cells at a 1:1 E to T ratio. HA or HI-specific scDbs were added at the indicated concentrations. After 72 hrs, supernatants from co-cultures were snap frozen and assayed for secreted MIP1β. FIG. 12B: T2 cells were pulsed with the ALT or ILK peptides at the indicated concentrations and 10 ug/ml B2M. Pulsed T2 cells were incubated with pre-activated CD8⁺T cells (1:2 E to T) and with 0.25 nM of HA29, HA122, and HI155-scDbs and 0.5 nM of HI12-scDb for 72 hrs. Co-culture supernatants were harvested and assayed for MIP1β. FIG. 12C: T2 cells were pulsed with indicated concentrations of the VLA peptide and co-cultured with pre-activated CD8⁺T cells in the presence of 25 ng/ml HV115. CTL activation in response to decreasing doses of peptide was measuring using surface CD69⁺ levels. FIG. 12D: No scDb.

FIGS. 13A-13B demonstrate the characterization of HA29-scDb binding. A library of 171 positional scanning variant peptides was generated by substituting each residue of the original peptide with the 19 other possible amino acids as previously described (38, 39). T2 cells were then pulsed with each variant at 10 uM and co-cultured with pre-activated CD8⁺ T cells from healthy donors. FIG. 13A: Supernatants were assayed for MIP1β at 24 hours, and the mean of three technical replicates is plotted as a heatmap. Black boxes indicate the amino acids in the parental peptides. FIG. 13B: To assess for differential peptide affinity for A2 and peptide-induced A2 upregulation on T2 cells post-pulsing, we also stained for surface HLA-A2 levels.

FIGS. 14A-14B demonstrate that HA29 and HI12-scDbs induce polyfunctional CD8⁺T cell responses. T2 cells were pulsed with the ALT or ILK peptides at the indicated concentrations. 10×10⁴ peptide-pulsed cells were co-cultured with 5×10⁴ pre-activated CD8⁺T cells (1:2 E to T) in the presence of 0.25 nM HA29 or HI12 scDbs for 72 hrs. Supernatants from co-cultures were assayed for the indicated effector molecules using Legendplex. Specific activation of CD8⁺T cells by HA29 and HI12 was observed in response to cells pulsed with the cognate but not irrelevant pMHC at doses down to the nanomolar range.

FIGS. 15A-15D demonstrate that HA29-scDbs induce viral suppression even in infected cells with A2 downregulation. FIG. 15A: Suppression assays were set up using target cells (activated CD4⁺T cells from an A2-expressing healthy donor) infected with ΔEnv NL4.3 EGFP and co-cultured with autologous pre-stimulated CD8⁺T cells for 72 hrs (see Methods) in the presence of HA29 or H2-scDb. A) MFI of surface HLA-A2 (BB7.2 clone) levels are shown from GFP+ or GFP− cells from two biological replicates of infected cells used in suppression assays. FIG. 15B: Representative flow cytometry plots of residual viable A2+ GFP⁺ cells after 3 days of co-culture in a suppression assay, showing a dose-dependent decrease with the HA29-scDb but no decrease with the H2-scDb. FIG. 15C: Healthy donor HLA-A*02:01⁺ CD4⁺T cells were activated with CD3/CD28 Dynabeads for 72 hrs and infected with ΔEnv NL4.3 EGFP. Two days post infection, infected or uninfected CD4⁺T cells were co-cultured at a 1:3 E:T ratio with autologous human CD8⁺T cells (15×10⁴ targets and 5×10⁴ CD8⁺T cells) with the HA29-scDb at 25 ng/ml for 72 hr. scDb-induced CD8⁺T cell activation was measured by MIP1β ELISAs from co-culture supernatants. FIG. 15D: % Viable cells as determined by flow cytometry in uninfected co-cultures to assess for background killing by increasing doses of HA29-scDb. Data in C show mean±SD analyzed by two-way ANOVA followed by Tukey's Multiple Comparison Test. Data in D represent the mean±SD of three independent experiments analyzed by one-way ANOVA followed by Dunnett's multiple comparison test. *p<0.05, **p<0.01, ***p<0.001, ****p<0.0001.

### DETAILED DESCRIPTION

Here we describe an approach to target HIV-1 peptide:MHC complexes (pMHC) using TCR mimic (TCRm) bispecific antibodies capable of linking CTLs to infected target cells and promoting target cell lysis. This approach differs from related approaches based on soluble TCRs in that phage-display was used to identify antibody-like reagents that bind with high affinity to HIV-1 pMHC complexes. Thus, this approach does not suffer from the limitations in affinity characteristic of TCR-based approaches. Phage screening identified single chain variable fragments (scFvs) with much higher affinity for target pMHC (38-40). Here, we generated novel bispecific TCRm reagents against CD3 and HIV-1 peptides bound to human leukocyte antigen (HLA) HLA-A*02:01 (A2) complexes, an allele present in >40% of the US Caucasian population (41), and evaluated their ability to mediate killing of infected cells. These antibodies utilize the sensitivity of T cell signaling as readouts for antigen processing and as agents to promote killing of infected cells. These scDbs are exquisitely sensitive and specific for the peptide portion of the pMHC. Most importantly, one scDb caused killing of infected cells presenting a naturally processed target pMHC. This work lays the foundation for a novel therapeutic killing strategy towards elimination of the HIV-1 reservoir. Preferred systems are disclosed in Srona Sengupta et al., (2022). PNAS 119 (15) e2123406119, doi.org/10.1073/pnas.2123406119, incorporated herein by reference in its entirety.

Infection with HIV-1 remains a major public health problem affecting more than 35 million people worldwide and more than 1.2 million people in the United States. Combined antiretroviral therapy (cART) can achieve a “functional cure”, but HIV-1 resurgence in latently infected cells after cART withdrawal is a main obstacle to a permanent cure of HIV-1 infection. Current cART does not eliminate the integrated and transcriptionally silent HIV-1 provirus in latently infected cells. While the introduction of combined antiretroviral therapy (cART) has greatly improved survival rates among AIDS patients, a substantial portion of HIV-1 infected individuals remain at risk for the development of AIDS as a result of reactivation of latently infected cells, partly due to non-adherence to medication and emergence of drug resistant viruses.

Moreover, HIV-1 positive long term survivors continue to develop comorbidities including an accelerated aging process, neurocognitive disorders, heart failure, and others. Gradual reactivation of the integrated HIV-1 genome in latently infected cells can result in superactivation of the HIV-1 long-term repeats (LTR) and the initiation of the productive infection cycle.

Current antiretroviral therapy does not eliminate the integrated and transcriptionally silent HIV provirus in latently infected cells. A “shock and kill” (also called “kick and kill” or “reactivation and elimination”) strategy to eradicate HIV latent reservoir has been becoming very promising, wherein reactivation of latent proviruses allows clearance of latent cells by viral cytotoxicity and/or host immune defense. The concomitant antiretroviral treatment will prevent virus spread and block new infection (Sgarbanti and Battistini, 2014, Curr Opin Virol 3:394-401). Several reagents or small molecules, in particular, the HDAC inhibitors, have been developed to reactivate HIV latent reservoir, some of which have been used in clinical trials (Wei et al., 2014, PLoS Pathog 10:e1004071; Lucera et al., 2014, J Virol 88:10803-12; Spivak et al., Clin Infect Dis 58:883-90; Xing and Silicano, 2013, Drug Discov Today 18:541-51). However, the reactivation results are disappointing, likely due to insufficient reactivation, non-specific cell targeting and drug toxicity (Lucera et al., 2014, J Virol 88:10803-12; Spivak et al., Clin Infect Dis 58:883-90; White et al., 2015, Antiviral Res 123:78-85). For example, a recent report using a humanized HIV-1 latency mouse model demonstrated that only combined treatment with three well-established latency-reversing agents including the histone deacetylase inhibitor vorinostat (suberoylanilide hydroxamic acid, SAHA), the BET bromodomain protein inhibitor I-BET 151, and the immune modulatory anti-CTLA4 antibody, allows HIV upregulation to a sufficient level in HIV latent reservoir cells for the elimination by the broadly neutralizing anti-HIV antibodies (Halper-Stromberg et al., 2014, Cell 158:989-99). Multiple activating, latency-reversing agents at several signal pathways (Laird et al., 2015, J Clin Invest 125:1901-12) will definitely increase the toxicity to HIV-negative cells, similar to the chemotherapies for treatment of cancerous cells. Furthermore, repeated administration of these latency-reversing agents is required to maintain a continuous reactivation of HIV latent reservoir. Therefore, a better reactivator of latent HIV virus, which displays targeted cell specificity, sustained high efficiency and no/low cytotoxicity remains to be identified.

Herein, the production of bispecific T cell receptor (TCR)-mimic (TCRm) diabodies which specifically bind retroviral peptides complexed with a major histocompatibility complex molecule (pMHC) and a T cell signal transduction peptide allow for their use in assays to identify latency reversing agents (LRAs). Reactivation of latent HIV, by way of the present disclosure, allows for the clearance of infected cells by antiviral therapy and/or the host immune system.

### Compositions

Accordingly, in certain aspects, a composition comprises a T cell receptor (TCR)-mimic (TCRm) antibody having two antigen binding domains, wherein (i) a first binding domain specifically binds to an HIV peptide complexed with a major histocompatibility complex molecule (pMHC) and (ii) a second binding domain specifically binds to T cell signal transduction peptide. In certain embodiments, the HIV peptide is an HIV-1 peptide or an HIV-2 peptide. In certain embodiments, the HIV-1 peptide comprises Gag, Pol, Env, or Reverse Transcriptase (RT). In certain embodiments, the major histocompatibility complex (MHC) comprises MHC-I, MHC-II or human leukocyte antigen E (HLA-E). In certain embodiments, the pMHC comprises an HIV-1, Gag, Pol, Env, or Reverse Transcriptase (RT) peptide complexed to MHC-I or HLA-E. In certain embodiments, the T cell signal transduction peptide comprises a CD3 peptide. In certain embodiments, the CD3 peptide comprises CD3γ, CD3δ, CD3ε, or CD3ζ. In certain embodiments, the CD3 peptide is CD3ε. In certain embodiments, the antigen binding domains comprise: polyclonal antibodies or fragments thereof, monoclonal antibodies or fragments thereof, antigen-binding antibody fragments, (Fab) fragments, F(ab′)2 fragments, Fab′ fragments, Fv fragments, recombinant IgG (rIgG) fragments, variable heavy chain (VH) regions, single chain antibody fragments, single chain variable fragments (scFv), single domain antibodies, bispecific, antibodies, diabodies, single-chain diabodies (scDb), triabodies, and tetrabodies, tandem di-scFv, or tandem tri-scFvs. In certain embodiments, the antigen binding domains comprise single-chain diabodies (scDb).

Diabodies (Db) are bivalent or bispecific antibody fragments generated by the dimerization of two VH-VL or VL-VH fragments (Holliger, P., Prospero, T. D. and Winter, G. (1993) Proc. Natl Acad. Sci. USA, 90. 6444-6448). Bispecific diabodies are produced by heterodimerization of two fragments of the structure VH_{A}-VL_{B} and VH_{B}-VL_{A} expressed in the same cell. The original dimeric diabody format by joining the two fragments can include an additional middle linker (linker M) (Brüsselbach, S., et al. (1999) Tumor Targeting, 4, 115-123. Kipriyanov, S., et al. (1999) J. Mol. Biol., 293, 41-56). The overall structure of these single-chain diabody (scDb) molecules is VH_{A}-linkerA-VL_{B}-linkerM-VH_{B}-linkerB-VL_{A}, with linkers A and B consisting routinely of 5-6 residues and linker M of 15-20 residues. The middle linker (linker M) resembles those connecting the two variable domains in scFv molecules. Furthermore, this heterodimeric molecule can be designed with a different configuration (VL_{mAbA}-VH_{mAbB}/VL_{mAbB}-VH_{mAbA}) (Brinkmann U. and Kontermann R. E. MAbs. 2017; 9:182-212). Each variable domain is connected to another one by a short peptide linker (five amino acids). Similar to BiTEs, diabodies have two different antigen-binding sites.

Interaction of two of the polypeptide chains will produce two VL-VH pairings, forming two epitope binding sites, i.e., a bivalent molecule. Neither the VH or VL domain is constrained to any position within the polypeptide chain, i.e., restricted to the amino (N) or carboxy (C) terminus, nor are the domains restricted in their relative positions to one another, i.e., the VL domain may be N-terminal to the VH domain and vice-versa. The only restriction is that a complimentary polypeptide chain be available in order to form a functional diabody. Where the VL and VH domains are derived from the same antibody, the two complimentary polypeptide chains may be identical. For example, where the binding domains are derived from an antibody specific for epitope A (i.e., the binding domain is formed from a VL_{A}-VH_{A} interaction), each polypeptide will comprise a VH_{A} and a VL_{A}. Homodimerization of two polypeptide chains of the antibody will result in the formation two VL_{A}-VH_{A} binding sites, resulting in a bivalent monospecific antibody. Where the VL and VH domains are derived from antibodies specific for different antigens, formation of a functional bispecific diabody requires the interaction of two different polypeptide chains, i.e., formation of a heterodimer. For example, for a bispecific diabody, one polypeptide chain will comprise a VL_{A} and a VL_{B}; homodimerization of the chain will result in the formation of two VL_{A}-VH_{B} binding sites, either of no binding or of unpredictable binding. In contrast, where two differing polypeptide chains are free to interact, e.g., in a recombinant expression system, one comprising a VL_{A} and a VH_{B} and the other comprising a VL_{B} and a VH_{A}, two differing binding sites will form: VL_{A}-VH_{A} and VL_{B}-VH_{B}. For all diabody polypeptide chain pairs, the possibly of misalignment or mis-binding of the two chains is a possibility, i.e., interaction of VL-VL or VH-VH domains; however, purification of functional diabodies is easily managed based on the immunospecificity of the properly dimerized binding site using any affinity based method known in the are or exemplified herein, e.g., affinity chromatography.

In certain embodiments, the one or more of the polypeptide chains of the diabody comprises an Fc domain. Fc domains in the polypeptide chains of the diabody molecules preferentially dimerize, resulting in the formation of a diabody molecule that exhibits immunoglobulin-like properties, e.g., Fc-FcγR, interactions. Fc comprising diabodies may be dimers, e.g., comprised of two polypeptide chains, each comprising a VH domain, a VL domain and an Fc domain. Dimerization of the polypeptide chains results in a bivalent diabody comprising an Fc domain, albeit with a structure distinct from that of an unmodified bivalent antibody. Such diabody molecules will exhibit altered phenotypes relative to a wild-type immunoglobulin, e.g., altered serum half-life, binding properties, etc. In other embodiments, diabody molecules comprising Fc domains may be tetramers. Such tetramers comprise two ‘heavier’ polypeptide chains, i.e. a polypeptide chain comprising a VL, a VH and an Fc domain, and two ‘lighter’ polypeptide chains, i.e., polypeptide chain comprising a VL and a VH. The lighter and heavier chains interact to form a monomer, and the monos interact via their unpaired Fc domains to form an Ig-like molecule. Such an Ig-like diabody is tetravalent and may be monospecific, bispecific or tetraspecific.

At least two binding sites of the diabody molecule can recognize the same or different epitopes. Different epitopes can be from the same antigen or epitopes from different antigens. In one embodiment, the epitopes are from different cells. In another embodiment, the epitopes are cell surface antigens on the same cell or virus. The epitopes binding sites can recognize any antigen to which an antibody can be generated. For example, proteins, nucleic acids, bacterial toxins, cell surface markers, autoimmune markers, viral proteins, drugs, etc. In particular aspects, at least one epitope binding site of the diabody is specific for an antigen on a particular cell, such as a B-cell or T-cell, a phagocytotic cell, a natural killer (NK) cell or a dendritic cell.

Each domain of the polypeptide chain of the diabody, i.e., the VL, VH domain may be separated by a peptide linker. The peptide linker may be 0, 1, 2, 3, 4, 5, 6, 7, 8, or 9 amino acids.

In certain embodiments, each polypeptide chain of the diabody molecule is engineered to comprise at least one cysteine residue that will interact with a counterpart at least one cysteine residue on a second polypeptide chain of the disclosure to form an inter-chain disulfide bond. The interchain disulfide bonds serve to stabilize the diabody molecule, improving expression and recovery in recombinant systems, resulting in a stable and consistent formulation as well as improving the stability of the isolated and/or purified product in vivo. The at least one cysteine residue may be introduced as a single amino acid or as part of larger amino-acid sequence, e.g. hinge domain, in any portion of the polypeptide chain.

In certain embodiments, the diabody molecule comprises at least two polypeptide chains covalently linked by a disulfide bond between the cysteine residues. In yet other embodiments, the at least one cysteine residue is engineered to occur at the N-terminus of the amino acid chain. In still other embodiments, the at least one cysteine residue is engineered to occur in the linker portion of the polypeptide chain of the diabody molecule. In further embodiments, the VH or VL domain is engineered to comprise at least one amino acid modification relative to the parental VH or VL domain such that the amino acid modification comprises a substitution of a parental amino acid with cysteine.

Single-chain diabodies (scDb) are readily assembled in bacterial and mammalian cells and show improved stability under physiological conditions. A detailed description of the production of diabodies provided herein is described in the examples section which follows.

In some embodiments, molecules of the disclosure are engineered to comprise an altered glycosylation pattern or an altered glycoform relative to the comparable portion of the template molecule. Engineered glycoforms may be useful for a variety of purposes, including, but not limited to, enhancing effector function. Engineered glycoforms may be generated by any method known to one skilled in the art, for example by using engineered or variant expression strains, by co-expression with one or more enzymes, for example, DI N-acetylglucosaminyltransferase III (GnTI11), by expressing a diabody of the disclosure in various organisms or cell lines from various organisms, or by modifying carbohydrate(s) after the diabody has been expressed and purified. Methods for generating engineered glycoforms are known in the art (Umana et al. (1999) Nat. Biotechnol 17:176-180, Davies et al. (2001) Biotechnol Bioeng 74:288-294; Shields et al. (2002) J Biol Chem 277:26733-26740; Shinkawa et al (2003) J Biol Chem 278:3466-3473; U.S. Pat. No. 6,602,684; Potillegent™ technology (Biowa, Inc Princeton, NJ), GlycoMAb™ glycosylation engineering technology (GLYCART biotechnology AG, Zurich, Switzerland).

The disclosure further encompasses incorporation of unnatural amino acids to generate the diabodies of the disclosure. Such methods are known to those skilled in the art such as those using the natural biosynthetic machinery to allow incorporation of unnatural amino acids into proteins, see. e.g., Wang et al. (2002) Chem. Comm. 1: 1-11; Wang et al. (2001) Science, 292: 498-500; van Hest et al. (2001) Chem. Comm. 19: 1897-1904. Alternative strategies focus on the enzymes responsible for the biosynthesis of amino acyl-tRNA, see, e.g., Tang et al. (2001) J. Am. Chem. Soc. 123(44): 11089-11090; Kiick et al. (2001) FEBS Lett. 502(1-2):25-30.

In some embodiments, the disclosure encompasses methods of modifying a VL, VH domain of a molecule of the disclosure by adding or deleting a glycosylation site. Methods for modifying the carbohydrate of proteins are well known in the art and encompassed within the disclosure. See, e.g., U.S. Pat. Nos. 6,218; 6,218,149; 6,472,511.

The diabodies used in the methods of the disclosure include derivatives that are modified, i.e., by the covalent attachment of any type of molecule to the diabody. For example, but not by way of limitation, the diabody derivatives include diabodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

For some uses, including in vivo use of antibodies in humans and in vitro detection assays, it may be preferable to use diabodies with variable domains derived from human, chimeric or humanized antibodies. Variable domains from completely human antibodies are particularly desirable for therapeutic treatment of human subjects. Human antibodies can be made by a variety of methods known in the art including phage display methods described above using antibody libraries derived from human immunoglobulin sequences.

A humanized antibody is an antibody, a variant or a fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody may comprise substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence.

Linkers: In certain embodiments, the bispecific antigen binding domains of the scDbs of the disclosure comprise one or more linkers. In certain embodiments, the linkers separate each of the antigen binding domains. Since the single chain diabodies comprise a VL and VH domain of an antibody contained in a single polypeptide chain the domains are separated by a flexible linker of sufficient length to allow self-assembly of the two domains into a functional epitope binding site.

Conjugation between each of the bispecific antigen domains can also be performed using a variety of chemical linkers. For example, each monovalent binding entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Covalent conjugation can either be direct or via a linker. In certain embodiments, direct conjugation is by construction of a protein fusion (i.e., by genetic fusion of the two genes encoding the pMHC and CD3 and expressed as a single protein). In certain embodiments, direct conjugation is by formation of a covalent bond. In certain embodiments, direct conjugation is by modification (i.e., genetic modification) of one of the two molecules to be conjugated to include a reactive group (as non-limiting examples, a sulfhydryl group or a carboxyl group) that forms a covalent attachment to the other molecule to be conjugated under appropriate conditions. Methods for covalent conjugation of nucleic acids to proteins are also known in the art (i.e., photocrosslinking, see, e.g., Zatsepin et al. Russ. Chem. Rev. 74: 77-95 (2005)).

Conjugation may also be performed using a variety of linkers. For example, a monovalent binding entity and a effector entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine.

The present disclosure also encompasses diabody molecules conjugated to a diagnostic or therapeutic agent or any other molecule for which serum half-life is desired to be increased/decreased and/or targeted to a particular subset of cells. The molecules of the disclosure can be used diagnostically to, for example, monitor the viral infection and reactivation of latent virus. Detection can be facilitated by coupling the molecules of the disclosure to a detectable substance or by the molecules immunospecifically recognizing the detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the molecules of the disclosure or indirectly, through an intermediate (such as, for example, a linker known in the art) using techniques known in the art, or the molecule may immunospecifically recognize the detectable substance: immunospecifically binding the substance. See, for example, U.S. Pat. No. 4,741,900 for metal ions which can be conjugated to antibodies for use as diagnostics according to the present disclosure. Such diagnosis and detection can be accomplished designing the molecules to immunospecifically recognize the detectable substance or by coupling the molecules of the disclosure to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol: bioluminescent materials such as, but not limited to, luciferase, luciferin, and aequorin: radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthenium (⁹⁷Ru), samarium (¹⁵³Sn), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions

### Methods of Treating

The molecules of the disclosure may be administered in combination with other therapies known to those skilled in the art for the treatment or prevention of HIV including but not limited to, current standard and experimental chemotherapies, anti-viral therapies, hormonal therapies, biological therapies, immunotherapies, radiation therapies, or surgery.

One or more molecule of the disclosure can be administered to a mammal, preferably a human, concurrently with one or more other therapeutic agents useful for the treatment of cancer. The term “concurrently” is not limited to the administration of prophylactic or secondary therapeutic agents at exactly the same time, but rather it is meant that a molecule of the disclosure and the other agent are administered to a mammal in a sequence and within a time interval such that the molecule of the disclosure can act together with the other agent to provide an increased benefit than if they were administered otherwise. For example, each prophylactic or other therapeutic agents may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. The prophylactic or other therapeutic agents can be administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. Two or more components can be administered within the same patient visit.

The prophylactic or other therapeutic agents can be administered at about 2 to 4 days apart, at about 4 to 6 days apart, at about 1 week part, at about 1 to 2 weeks apart, or more than 2 weeks apart. The prophylactic or other therapeutic agents can be administered in a time frame where both agents are still active. One skilled in the art would be able to determine such a time frame by determining the half life of the administered agents.

The prophylactic or other therapeutic agents of the disclosure can be cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

Prophylactic or other therapeutic agents can be administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

The therapeutic and prophylactic agents of the disclosure can be administered in metronomic dosing regimens, either by continuous infusion or frequent administration without extended rest periods. Such metronomic administration can involve dosing at constant intervals without rest periods. Typically the therapeutic agents, in particular cytotoxic agents, are used at lower doses. Such dosing regimens encompass the chronic daily administration of relatively low doses for extended periods of time. In preferred embodiments, the use of lower doses can minimize toxic side effects and eliminate rest periods. In certain embodiments, the therapeutic and prophylactic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months. The scheduling of such dose regimens can be optimized by the skilled oncologist.

Courses of treatment can be administered concurrently to a mammal, i.e., individual doses of the therapeutics are administered separately yet within a time interval such that molecules of the disclosure can work together with the other agent or agents. For example, one component may be administered one time per week in combination with the other components that may be administered one time every two weeks or one time every three weeks. In other words, the dosing regimens for the therapeutics can be carried out concurrently even if the therapeutics are not administered simultaneously or within the same patient visit.

When used in combination with other prophylactic and/or therapeutic agents, the molecules of the disclosure and the prophylactic and/or therapeutic agent can act additively or, more preferably, synergistically. A molecule of the disclosure can be administered concurrently with one or more therapeutic agents in the same pharmaceutical composition. A molecule of the disclosure can be administered concurrently with one or more other therapeutic agents in separate pharmaceutical compositions. A molecule of the disclosure can be administered prior to or subsequent to administration of another prophylactic or therapeutic agent. Administration of a molecule of the disclosure in combination with other prophylactic or therapeutic agents can be by the same or different routes of administration, e.g., oral and parenteral. In certain embodiments, when a molecule of the disclosure is administered concurrently with another prophylactic or therapeutic agent that potentially produces adverse side effects including, but not limited to, toxicity, the prophylactic or therapeutic agent can advantageously be administered at a dose that falls below the threshold that the adverse side effect is elicited.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the route of administration, as well as age, body weight, response, and the past medical history of the patient. Suitable regimens can be selected by one skilled in the art by considering such factors and by following, for example, dosages reported in the literature and recommended in the Physician's Desk Reference (56th ed., 2002).

Accordingly, in certain embodiments, a method of treating a subject diagnosed with a human immunodeficiency virus (HIV) infection, comprises administering to the subject a therapeutically effective amount of a bispecific T cell receptor (TCR)-mimic (TCRm) antibody comprising two binding domains, wherein (i) a first binding domain specifically binds to an HIV peptide complexed with a major histocompatibility complex molecule (pMHC) and (ii) a second binding domain which specifically binds to T cell signal transduction peptide. In certain embodiments, the HIV peptide is an HIV-1 peptide or an HIV-2 peptide. In certain embodiments, the HIV-1 peptide comprises Gag, Pol, Env, or Reverse Transcriptase (RT). In certain embodiments, the major histocompatibility complex (MHC) MHC-I, MHC-II or human leukocyte antigen E (HLA-E). In certain embodiments, the pMHC comprises an HIV-1, Gag, Pol, Env, or Reverse Transcriptase (RT) peptide complexed to MHC-I or HLA-E. In certain embodiments, the T cell signal transduction peptide comprises a CD3 peptide. In certain embodiments, the CD3 peptide comprises CD3γ, CD3δ, CD3ε, or CD3ζ. In certain embodiments, the CD3 peptide is CD3ε. In certain embodiments, the antigen binding domains comprise single-chain diabodies (scDb). In certain embodiments, the method further comprises administering one or more secondary therapeutics. In certain embodiments, the secondary therapeutics comprise anti-viral agents, gene-editing agents, latency reversing agents (LRAs) or combinations thereof.

In certain embodiments, the TCRm antibodies embodied herein are administered to a patient in combination with one or more other anti-viral agents or therapeutics. Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

In certain embodiments, the TCRm antibodies embodied herein are administered with one or more compositions comprising a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, a composition comprises a therapeutically effective amount of at least one NNRTI or a combination of NNRTI's, analogs, variants or combinations thereof. In certain embodiments, the NNRTI is rilpivirine. In certain embodiments, an NRTI comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof. In certain embodiments, the composition comprises a therapeutically effective amount of at least one or a combination of NRTI's, analogs, variants or combinations thereof.

In certain embodiments, the TCRm antibodies embodied herein are administered with one or more latency reversing agents (LRAs). The LRAs can be any known LRAs or can be identified using an assay provided herein. The assay comprises producing bispecific diabodies embodied herein, which specifically bind to human immunodeficiency virus (HIV) peptides complexed with MHC molecules (pMHC), screening HIV infected cells against a library of candidate agents; contacting the HIV infected cells with the pMHC specific diabodies; assaying for changes in expression or levels of pMHC antigens and/or an assay measuring virion production; thereby identifying latency reversing agents. In certain embodiments, the screening assay is a high-throughput screening assay.

Various quantification protocols can be used to determine the viral growth over time, but assays quantifying the amount of viral antigen (p24 ELISA) and enzyme activity (RT activity) are the most common (Oberle C. S. et al. Retrovirology. 2016; 13 doi: 10.1186/s12977-016-0299-0 Kiguoya M. W., et al. J. Virol. 2017; 91 doi: 10.1128/JVI.00253-17). More recently, protocols using TZM-bl cells that express luciferase under the control of the HIV-1 Tat promotor, have been used to determine viral infectivity, such as determination of the 50% tissue culture infectious dose (TCID₅₀) (Ojwach D. B. A., et al. J. Virol. 2018; 92 doi: 10.1128/JVI.00811-18). Furthermore, parallel infection assays do not require modifications of the viruses and/or cells, which makes them cost effective and suitable for high throughput, large scale experiments. There are established assays that can determine replication capacities of large sample populations of recombinant viruses that consist of a specific part of the patients' viral genome (e.g., gag, pol) cloned into a control backbone (e.g., HIV-1 NL4-3).

The candidate LRAs can be small molecule compounds, antisense reagents, siRNA reagents, antibodies, enzymes, peptides organic or inorganic molecules, natural or synthetic compounds and the like.

In certain embodiments, the LRA is a gene-editing agent. For example, a composition for reactivation of a retrovirus in vitro or in vivo comprises an isolated nucleic acid encoding a guide nucleic acid, wherein the guide nucleic acid comprises a targeting nucleotide sequence directed to one or more target sequences in the retroviral genome; an isolated nucleic acid encoding a Clustered Regularly Interspaced Short Palindromic Repeat (CRISPR)-associated endonuclease/Cas (CRISPR/Cas) fusion protein, comprising CRISPR/Cas and one or more transcriptional activators; and, an isolated nucleic acid encoding a fusion protein comprising an RNA binding protein, fragments, mutants, derivatives or variants thereof and one or more transcriptional activators.

In certain embodiments, the CRISPR/Cas fusion protein comprises catalytically deficient Cas protein (dCas), orthologs, homologs, mutants variants or fragments thereof, fused with one or more transcriptional activators. In some embodiments, the one or more transcriptional activators comprise VP64, p65, HSF1, p65AD, Rta, Sp1, Vax, GATA4, fragments, mutants, or any combinations thereof.

In one embodiment, the present disclosure provides a method for the treatment or prevention of HIV infection in a subject in need thereof. For example, the present method allows for reactivation of latent HIV by one or more LRAs and administration of the TCRm antibodies. The reactivated HIV-infected cells can be eliminated by virus-induced cytotoxicity and/or the host immune system. In certain embodiments a secondary therapeutic agent may also be administered.

### Administration

Various delivery systems are known and can be used to administer a composition comprising the diabodies of the disclosure, e.g., encapsulation in liposomes, microparticles, microcapsules, recombinant cells capable of expressing the antibody or fusion protein, receptor-mediated endocytosis (See, e.g., Wu et al. (1987) J. Biol. Chem. 262:4429-4432), construction of a nucleic acid as part of a retroviral or other vector, etc. Methods of administering a molecule of the disclosure include, but are not limited to, parenteral administration (e.g., intradermal, intramuscular, intraperitoneal, intravenous and subcutaneous), epidural, and mucosal (e.g., intranasal, rectal and oral routes). In a specific embodiment, the molecules of the disclosure are administered intramuscularly, intravenously, rectally or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903.

The amount of the composition which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

For diabodies encompassed by the disclosure, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. The dosage and frequency of administration of diabodies of the disclosure may be reduced or altered by enhancing uptake and tissue penetration of the diabodies by modifications such as, for example, lipidation.

In one embodiment, the dosage of the molecules of the disclosure administered to a patient may be from 0.01 mg to 1000 mg/day when used as single agent therapy. In another embodiment the molecules of the disclosure are used in combination with other therapeutic compositions and the dosage administered to a patient are lower than when the diabodies are used as a single agent therapy.

Treatment of a subject with a therapeutically or prophylactically effective amount of molecules of the disclosure can include a single treatment or, preferably, can include a series of treatments. In a preferred example, a subject is treated with molecules of the disclosure in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. The pharmaceutical compositions described herein can be administered once a day, twice a day, or three times a day. The pharmaceutical compositions can be administered once a week, twice a week, once every two weeks, once a month, once every six weeks, once every two months, twice a year or once per year. It will also be appreciated that the effective dosage of the molecules used for treatment may increase or decrease over the course of a particular treatment.

### Pharmaceutical Compositions

The compositions described herein include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the disclosure comprise a prophylactically or therapeutically effective amount of one or more molecules and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: TCR-Mimic Bispecific Antibodies to Target the HIV-1 Reservoir

The oncology field provides examples of targeted therapies that enhance CD8⁺ T cell responses, resulting in successful treatment of certain cancers (26). Chimeric antigen receptor (CAR)-T cell approaches have received considerable attention (26-28) but require modification and re-infusion of patient-derived effector cells. In contrast, bispecific antibody engagers that link CTLs to antigen-bearing target cells and simultaneously activate cytolytic effector function do not require ex vivo manipulation of patient cells (29). Blinatumomab, a bispecific antibody against CD3 and CD19, allows CD3-expressing T cells to kill CD19-expressing B cell precursors and improves survival in relapsed or refractory acute lymphoblastic leukemia (ALL) (30). Early efforts to promote killing of HIV-1-infected cells using bifunctional molecules utilized immunotoxins targeting the HIV-1 Envelope (Env) proteins gp120 or gp41(31, 32). More recently, bispecific antibodies against Env and CD3 (33, 34) have yielded promising results in ex vivo latency clearance assays with cells from PLWH (34). However, the low level of Env expression on infected cells (35-37) and the extraordinary sequence variation in this protein highlight the need for alternative approaches.

Here we describe an approach to target HIV-1 peptide:MHC complexes (pMHC) using TCR mimic (TCRm) bispecific antibodies capable of linking CTLs to infected target cells and promoting target cell lysis.

### Materials and Methods

### Human Samples

Leukapheresis samples from HLA-A*02:01⁺ donors were obtained from Stem Cell Technologies, Vancouver, BC. PBMC were purified by density gradient centrifugation with Ficoll Paque Plus (GE) and cryopreserved.

### Cell Lines and Primary Cells

All cells were grown at 37° C. under 5% CO₂. PBMCs were thawed in RPMI 1640 with 10% FBS, 1% penicillin/streptomycin and rested overnight before stimulation with 15 ng/ml of anti-human CD3 antibody (clone OKT3, BioLegend) for three days in base media (RPMI 1640 with 10% FBS, 1% penicillin/streptomycin) containing 250 U IL-2 and 5 ng/mL IL-7 (hereafter termed IL-2/IL-7 media). OKT3 was removed after 3 days of activation and cells were maintained in IL-2/7 media at 1×10⁶ cells/ml for 7-10 days before isolation of CD8⁺T cells by negative selection (StemCell) for co-culture experiments. Separately, autologous PBMCs were also used to isolate CD4⁺T cells by negative selection (StemCell). CD4⁺T cells were activated using Human T-Activator CD3/CD28 Dynabeads (Thermo Fisher Scientific) in RPMI 1640 with Glutamax, 10% FBS, and 1% Pen/Strep and 30 U IL-2.

T2 cells were cultured in RPMI 1640+Glutamax with 10% FBS, 1% penicillin/streptomycin. RPMI 6666 cells were grown in the same media and used as an additional A2+ cell line for negative selection stages of phage panning.

### LC-DIAMS

LC-DIAMS requires fragmentation patterns and relative elution positions for all synthetic peptides which are predicted to bind to HLA-A*02:01 (Table 1). The pattern and elution map for each synthetic peptide can then be interrogated in complex samples containing minute quantities of the target peptide. For elution mapping, synthetic peptides were added to an extract of peptides from HeLa cells. Separately, peptides from HeLa digests were spiked into a sample containing A2-bound peptides eluted from W6/32 immunoprecipitation of CaSki, an HLA-A*02:01 expressing cell line. Both samples were run on LC-DIAMS. The relative position of synthetic peptides compared to HeLa peptides, and the relative position of HeLa peptides compared to endogenous A2*01-bound peptides, could be integrated to predict the elution position of synthetic HIV-1 epitopes among a background of endogenous A2*01 epitopes. These elution maps could be used to analyze whether low levels of candidate peptides were indeed presented on the surface of infected cells.

To prepare infected cells for LC-DIAMS, CD4⁺T cells from an HLA-A2+ healthy donor were activated with CD3/CD28 Dynabeads and infected with ΔEnv-NL4.3-EGFP. Three days after infection, cells were sorted into GFP+ and GFP− fractions and snap frozen. Frozen cells were lysed. MHC class I molecules were immunoaffinity purified with W6/32, and peptides were eluted from bead-bound pMHC-I using low pH. Peptide quantification of native pMHC by targeted Poisson detection was achieved by adding 100 attomoles of heavy (isotope-labeled) versions of ALT and VLA and 200 attomoles of heavy ILK to the GFP⁺ samples as previously described (43, 44).

Peptides and pHLAs

Peptides were synthesized at >95% purity (Elim Biopharm or JPT Peptide Technologies) with the exception of the crude peptides used for the positional scanning library and for LC-DIAMS. Peptides were resuspended in dimethylformamide (DMF) and stored at −80° C. HLA-A2 was refolded with peptide and beta-2 microglobulin, purified by gel filtration, and biotinylated (Fre Hutchinson Immune Monitoring Lab, Seattle, WA or Baylor MHC Tetraner Production Lab, Houston, TX). Confirmation of pMHC refolding before phage panning was done using ELISA with the antibody W6/32 (Biolegend).

### Phage Display Library Construction

The scFv-bearing library used for phage panning was previously described (38, 39, 51) and was regrown within a week of selection.

Phage Panning and Characterization and scDb Preparation

Phage panning was performed as previously described (38). Briefly, panning consisted of four rounds of negative and positive selection. Negative selection was performed against naked streptavidin beads, free streptavidin, and irrelevant, and biotinylated A2-pMHC pre-conjugated to streptavidin beads. Negative selection was also performed against A2-bearing cells (T2s, RPMI-6666s). Positive selection was performed for each round of panning with decreasing amounts of the relevant biotinylated monomer conjugated to streptavidin beads. After each round of panning, phage were eluted with glycine (pH 2), neutralized with Tris-HCL (pH 9), amplified in SS320 cells (Lucigen), and concentrated in PEG-NaCl. Phage eluted from the 4^{th} round of panning were diluted such that infection of bacteria produced single colonies. Individual bacterial colonies were inoculated in deep 96-well plates to produce monoclonal phage. Monoclonal phage supernatants were tested for their ability to bind to the target versus irrelevant pMHC using ELISA and flow cytometry. Phage supernatants were added to streptavidin ELISA plates pre-coated with biotinylated A2-monomers of the relevant or irrelevant pMHC, and phage binding was assessed using a rabbit anti-M13 antibody (Pierce) and a secondary conjugated to HRP. To assess phage binding to pMHC on the surface of cells, T2 cells were pulsed with 50 ug/ml peptide and 10 ug/ml B2M for 4 hrs to overnight at 37° C. in RPMI 1640+Glutamax with 1% Pen/Strep. Pulsed cells were washed once with PBS, incubated with monoclonal phage, and then stained with rabbit anti-M13 (Pierce) followed by a PE-conjugated donkey anti-rabbit secondary (Biolegend). Phage-stained cells were acquired on the Intellicyt flow cytometer and analyzed with FlowJo.

### Surface Plasmon Resonance (SPR) Affinity Measurements

SPR experiments were carried out on a Biacore T200 (Cytiva) at 25° C. of a SA chip. HBS-P (10 mM HEPES pH 7.4, 150 mM NaCl, 0.05% v/v surfactant P20) was used as the immobilization and capture running buffer. The three HILA ligands used were biotinylated A*02:01 bound to ILK, ALT, and VLA peptides. Approximately ~30 RU of each pMHC ligand was captured onto flow cells FCs 2 through 4 while flow cell FC1 was used as the reference subtraction. Bispecifics HA29 and HI12 were flown over as analytes at a rate of 50 μL/min in increasing concentration 3.125, 6.25, 12.5, 25, 50, 100, 200, and 400 nM, two-fold dilutions). The scDb HI12 was flowed over FC-2 (biotinylated HLA-A*02:01 bound to ILK) by increasing concentrations of 25, 50, 100, 200, and 400 nM (two-fold dilutions). Multi-cycle kinetics were performed in the presence of HBS-P supplemented with 5% glycerol with contact and dissociation times of 120 sec and 600 sec, respectively. One 20 sec injection of 2 M NaCl was used for surface regeneration. Binding responses for kinetic analyses were referenced and blank subtracted. All curves were fit with a 1:1 kinetic binding model using Biacore Insight Evaluation Software. All SPR measurements were done in duplicates.

scDb Expression and Purification

Monoclonal phages that stained specifically for a particular peptide-pulsed target were sequenced to identify the expressed Fab. Gene blocks expressing this Fab fragment, the UCHT1 Fab fragment against CD3, and C′terminal His tag were cloned via Gibson assembly into the pcDNA3.4 vector backbone. scDb constructs were sequenced, amplified, and expressed via transfection into 293T cells. scDbs were purified using nickel columns (Capturem His-tag miniprep columns) using 400 mM imidazole and were desalted to remove imidazole using Zeba Spin columns. scDbs were run on SDS-PAGE and quantified using iBright densitometric analysis in comparison to a BSA standard. Large-scale preparations of scDbs were produced by ThermoFisher.

pMHC ELISA Binding Assay

Monomers (1 ug/mL) in BAE blocking buffer (phosphate-buffered saline or PBS, 0.5% BSA, 0.1% sodium azide) were added to a EvenCoat streptavidin coated plate (R&D Systems) and incubated at 4° C. for 16h. Plate was washed with 1×TBS-T (1×TBS+0.05% Tween-20) using a BioTek 405 plate washer and scDbs (5 ug/mL in BAE) were plated and incubated at RT for 1 h. Plate was washed and HRP anti-6×His tag migG secondary (clone J099B12, Biolegend, 0.5 ug/mL in BAE) was plated and incubated at RT for 1h. Plate was washed and developed in TMB for 5 min at RT before stopping in 1N sulfuric acid. OD450 was read. All conditions were tested in triplicate, average and SD shown. For measuring the limit of scDb binding, binding was conducted as above with decreasing concentrations of scDb as indicated.

### Peptide Titration Co-Cultures

T2 cells were pulsed overnight with varying concentrations of peptide (see figures) in RPMI media containing 10 ug/mL beta-2 microglobulin (B2M) and 1% Penicillin/Streptomycin. Peptide-pulsed T2 cells were co-cultured with pre-activated healthy donor CD8⁺ T cells at a 1:2 E:T ratio in 10% FBS 1% Penicillin/Streptomycin RPMI in a 96-well V-bottom plate. All scDbs were added to a concentration of 0.25 nM. Supernatants were collected after 3 days of co-culture. Human CCL4/MIP-1B Quantikine ELISA (R&D Systems) and Human CD8/NK Panel Legendplex (Biolegend) were performed as per the manufacturers' protocols.

### Positional Scanning Variant Testing

171 positional scanning variant peptides were obtained by substituting each residue of the original peptide with the 19 other possible amino acids as previously described (39). T2 cells were pulsed with 10 ug/ml B2M and each variant peptide at 10 uM in serum-free RPMI for 4 hours. Pulsed cells were co-cultured 1:1 with pre-activated CD8⁺T cells from healthy donors and 0.25 nM HA29-scDb. Co-culture supernatants were assayed for MIP1β (Human CCL4/MIP-1B Quantikine ELISA, R&D Systems) at 24 hours. HLA-A2 levels on pulsed cells were also assessed by surface staining with an anti-HLA-A2 antibody (Biolegend) and viability dye (eFluor 780, ThermoFisher).

### CFSE and CellTraceViolet (CTV) Dual Stain Co-Culture

T2 cells were pulsed with 1 ug/ml peptide and 10 ug/ml β2M in serum-free RPMI for 4 hr at 37° C. ALT- or ILK-pulsed cells were counted, pelleted, and stained with CellTraceViolet (ThermoFisher) or CFSE (ThermoFisher), respectively, as per manufacturer's instructions. Stained cells were washed 3 times with excess PBS. 15×10⁴ pre-activated CD8⁺T cells were then combined with 5×10⁴ cells from each target population in 96-well V-bottom plates (3:1:1). HA29 and HI12 were added to respective wells at 4, 8, or 12 pM. Control wells contained the HLA-A2-scDb (pan A2, clone BB7.2) or isotype control H2-scDb (39) at 0.25 nM. Precision count beads (Biolegend) were added to each well for quantification of absolute cell numbers. Cells were pelleted and after incubation at 37° C. for 18 hrs, co-cultures were stained with Fixable Viability Dye eFluor 780 (ThermoFisher). Samples were acquired on the Intellicyt and analyzed by FlowJo (Treestar).

The count of viable ALT-pulsed (CFSE^{hi}) or ILK-pulsed (CTV^{hi}) cells remaining after co-culture was obtained by normalizing to the number of cells to the number of Precision Count Beads identified in the relevant well. For example, if 20,000 beads were plated and 10,000 beads were recovered upon flow cytometry acquisition, then the absolute count of cells in the well would be twice the amount in the flow gate. The % reduction or cell death of a specific target was obtained by the following formula (shown for ALT-pulsed cells): (CFSE^{hi} cells without scDb-CFSE^{hi} cells with HA29-scDb)/(CFSE^{hi} cells without scDb×100).

### In Vitro Infection Suppression Assay

On day −10, PBMCs from healthy donors were stimulated with anti-human CD3 antibody (clone OKT3, BioLegend) for three days, then maintained in RPMI 1640 with 10% FBS, 1% penicillin/streptomycin, 250 U IL-2, and 5 ng/mL IL-7. On day −3, the CD8⁺T cells were isolated from the stimulated PBMCs (StemCell). CD4⁺ T cells were also isolated from unstimulated, autologous PBMCs on Day −3 (StemCell) and activated with CD3/CD28 Dynabeads (ThermoFisher). On day 0, activated CD4⁺ T cells were spinoculated with single-round ΔEnv-NL4.3-EGFP with ×4 Env at 400 ng p24/100,000 cells at 800 g for 2 hrs at 37° C. Infected and uninfected (pseudospinoculated) cells were rested at 37° C. for 2.5 hours after spinoculation. scDbs were plated in 96-well U-bottom plates and preincubated with CD8⁺ T cells for 1 hour in conditioned T cell media (STCM) prior to co-culturing with CD4⁺ T cells at a 3:1 effector-to-target ratio for 3 days at 37° C. On day 3, culture supernatant was assayed for MIP1β using the Human CCL4/MIP-1B Quantikine ELISA (R&D Systems). Precision Count Beads (BioLegend) were added to sample wells. Cells were stained with Fixable Viability Dye eFluor 780 (ThermoFisher), Brilliant Violet 605 anti-human CD8 Antibody (BioLegend), and Brilliant Violet 421 anti-human CD3 Antibody (BioLegend) for 15 minutes at 4° C. After washing cells, samples were acquired on an Intellicyt flow cytometer and analyzed with FlowJo software.

A similar procedure was used for suppression assays involving the 33A10 patient isolate. Activated CD4⁺ T cells from an HLA-A2⁺ healthy donor were spinoculated with 33A10 as above. Infected and uninfected (pseudo-spinoculated) cells were rested, washed with STCM, and then co-cultured with autologous CD8⁺T cells (prepared as above) at a 3:1 E:T ratio. The HA29, HI12, and H2-scDbs were added to the co-cultures at 0.25 mM. Plates were spun at 800 RPM for 1 min and incubated for 3 days at 37° C. 30 ul supernatant were collected on Days 1, 3, and 5 for measurement by p24 ELISA (PerkinElmer).

### Statistical Analysis

Statistical analyses were carried out using the tests indicated in the figure legends. A P value of <0.05 is considered statistically significant. Analysis was performed using Prism Version 9 (GraphPad).

### Results

### Identification of A2-Restricted CTL Epitopes Physically Presented on Infected CD4⁺T Cells.

HIV-1 pMHC-I complexes on infected cells serve as ideal targets for antibody-based retargeting strategies. Epitopes can be identified based on predicted MHC I binding (42) and functional studies with synthetic peptides, but sequencing of peptides from purified class I molecules provides the strongest evidence that a particular p:MHC-I complex is present on infected cells. The identification of minute numbers of viral peptides within a vast excess of self-peptides has been challenging using traditional data-dependent mass spectrometry, which relies on peak intensities to identify peptide hits. Therefore, we utilized a highly sensitive Poisson detection liquid chromatography data independent acquisition mass spectrometry (LC-DIAMS) method (43, 44). LC-DIAMS relies on in silico algorithms to predict putative high-affinity CTL epitopes binding to MHC alleles of interest (45). These peptides are then synthesized and their fragmentation patterns and elution positions relative to a set of retention time (RT) peptides are measured and archived. These features are used by the Poisson algorithm to identify the elution and fragmentation of low-levels of the target peptide against a complex background of endogenous peptides. Identified peptides can then be quantitated in a subsequent run by adding isotope-labeled peptide analogs prior to acid elution such that subsequent processing steps are shared. If native Western blots demonstrate no HLA in the clearing pellet and complete HLA depletion from the soluble lysate, all HLA complexes are on the beads and relative ion peak intensities between the native and labeled peptides convey relative abundances.

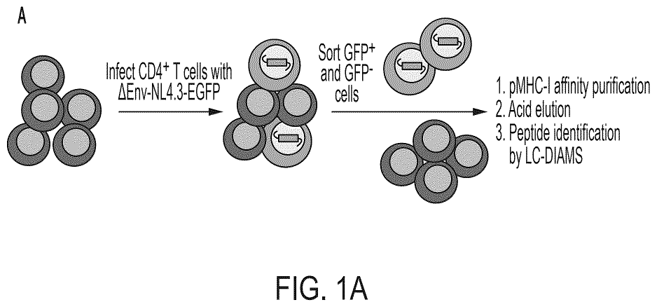

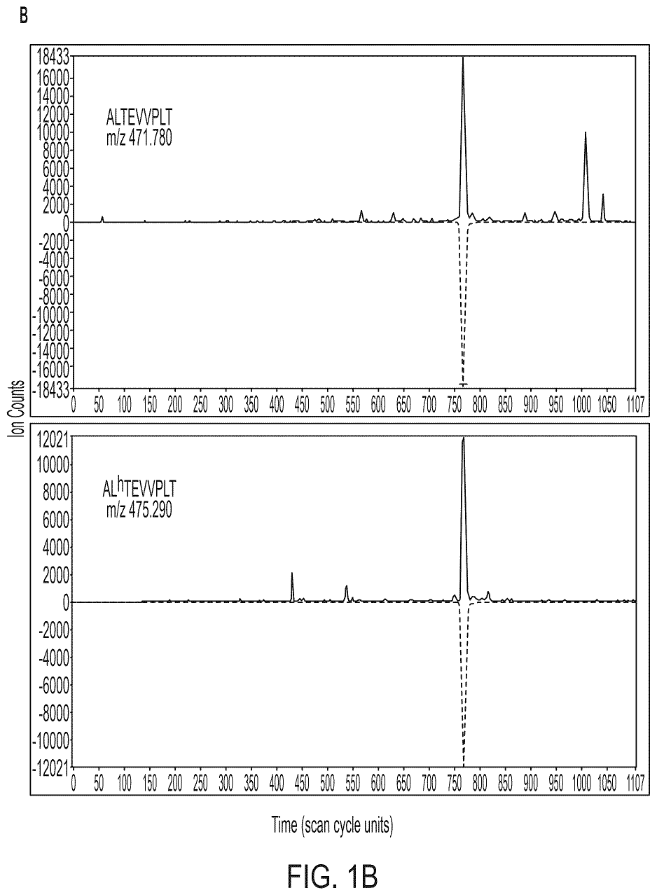

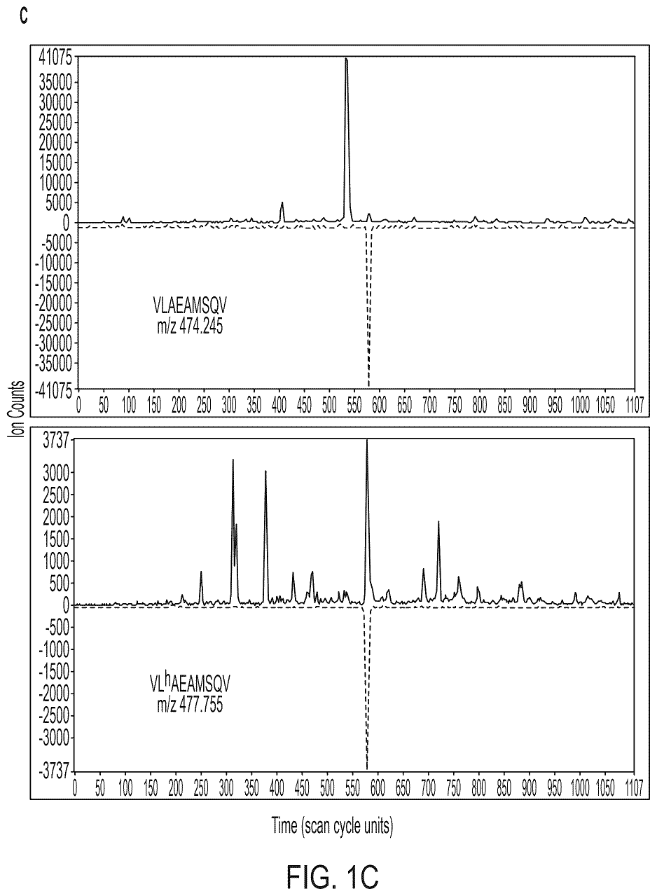

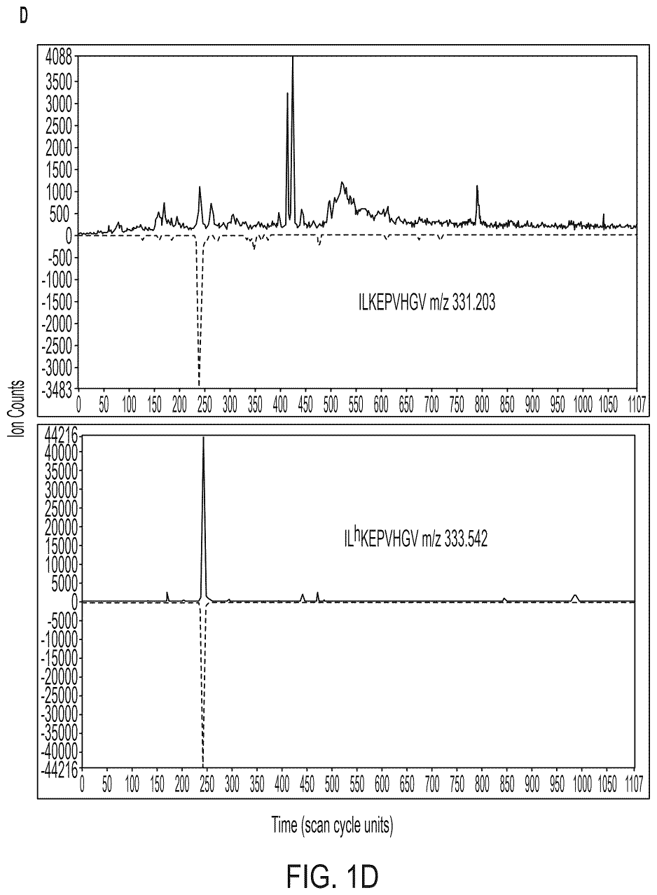

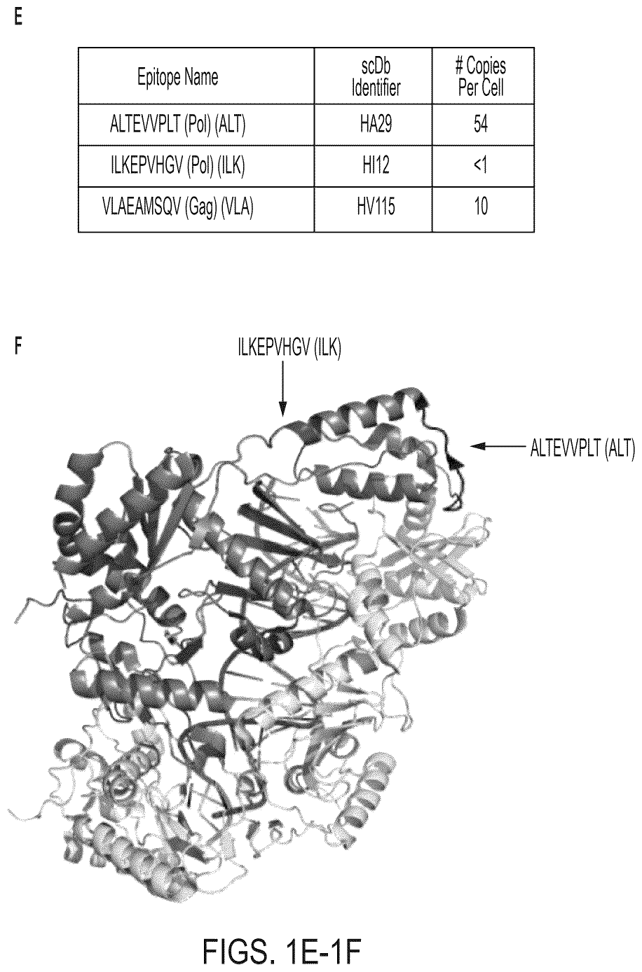

Across the HIV-1 proteome (excluding the variable Env protein), 107 peptides were predicted to bind to HLA-A*02:01 with high affinity and were synthesized (Table 1). Fragmentation patterns could be identified for 64 peptides, with cysteine-containing peptides and very hydrophobic peptides explaining most of the misses. We applied LC-DIAMS to identify HLA-A*02:01 restricted epitopes presented on CD4⁺T cells that had been infected in vitro with a GFP-tagged HIV-1 that is capable of a single-round infection (hereafter referred to as ΔEnv-NL4.3-EGFP) (46). Briefly, CD4⁺T cells from an HLA-A2⁺ healthy donor were activated with CD3/CD28 Dynabeads and infected with ΔEnv-NL4.3-EGFP. On day 3, cells were sorted into GFP⁺ and GFP⁻ fractions and snap frozen (FIG. 1A). Frozen cells were lysed, and class I MHC molecules were immunoaffinity purified with the pan-MHC I antibody W6/32. Peptides were eluted from bead-bound pMHC using low pH for subsequent LC-DIAMS analysis.

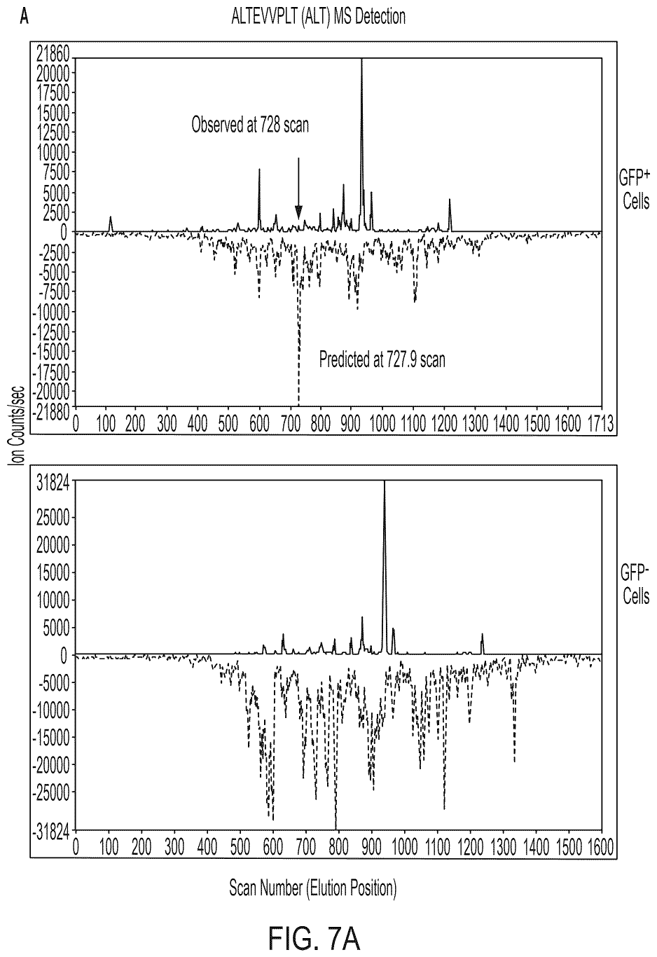

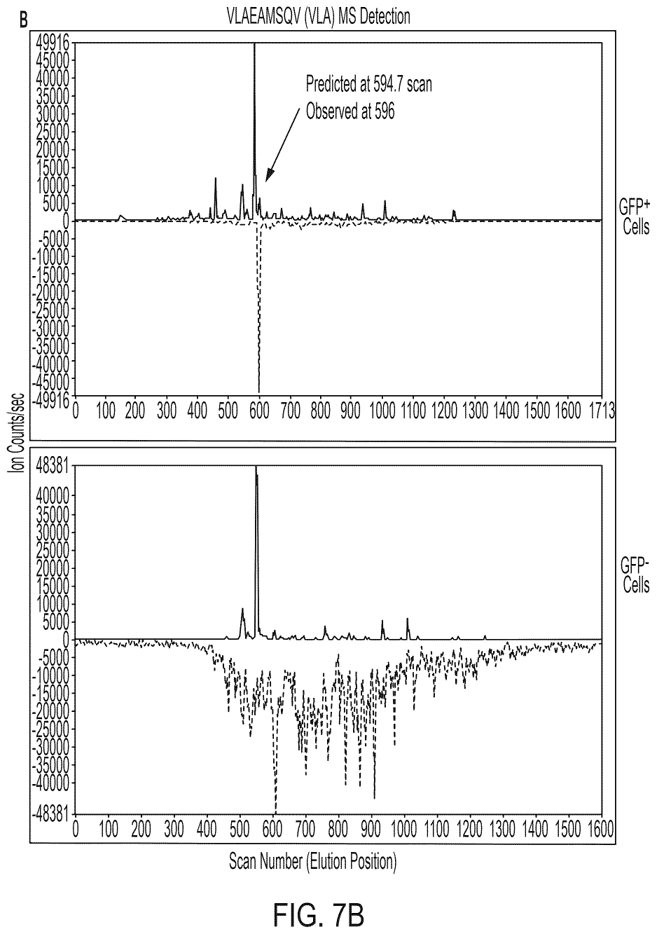

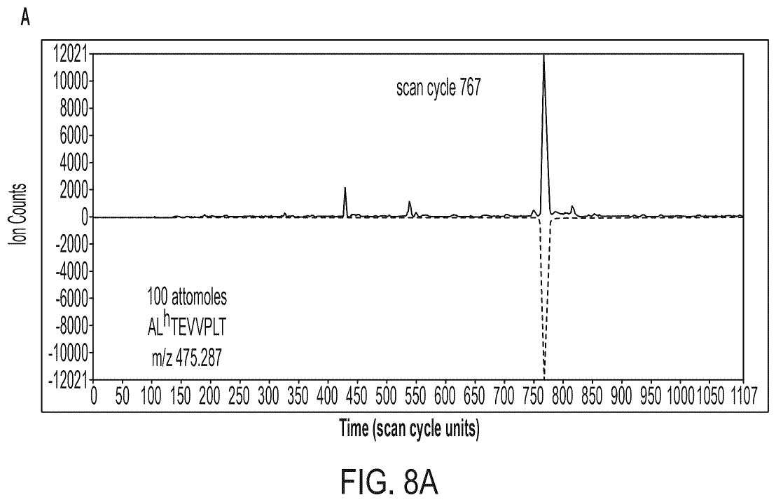

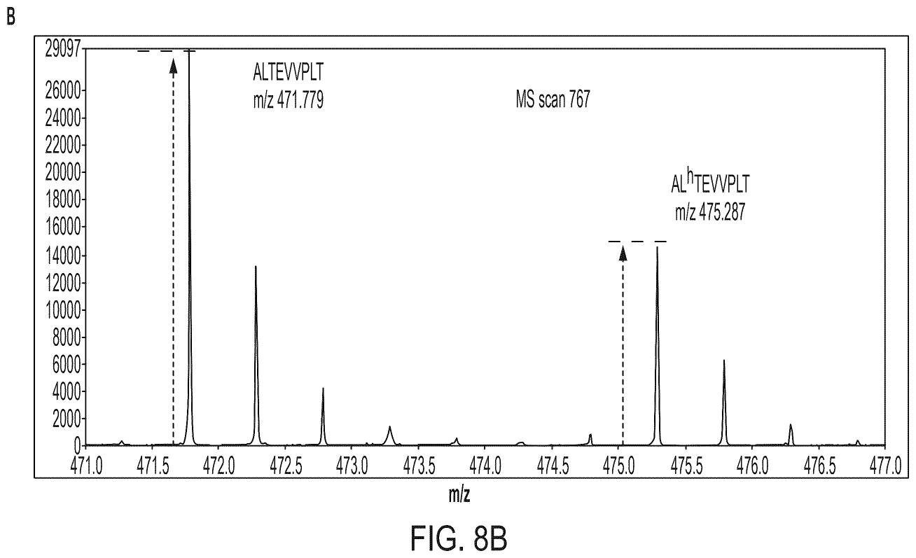

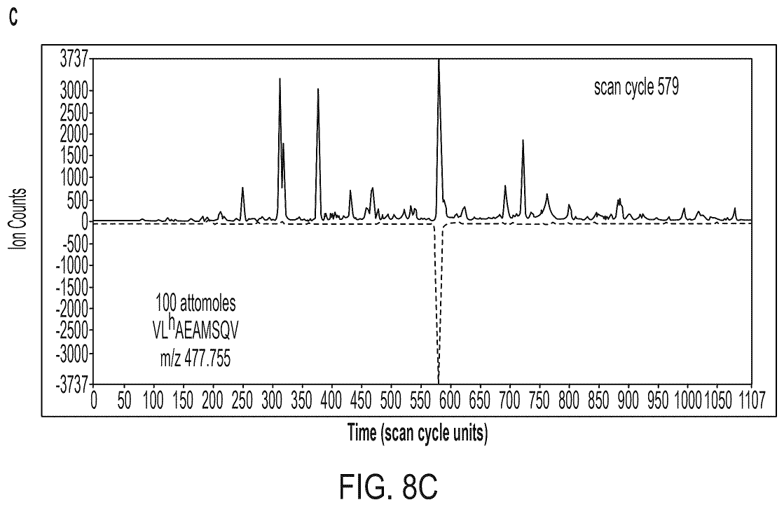

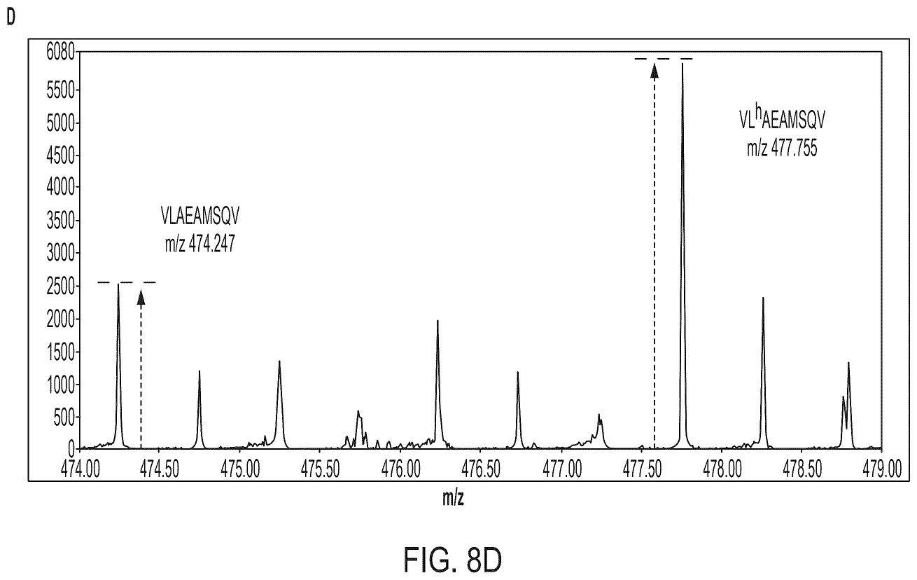

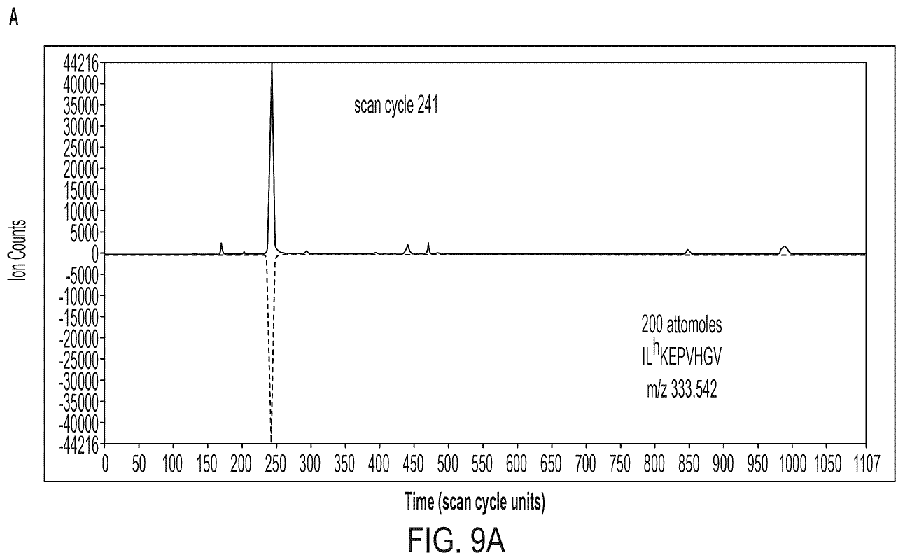

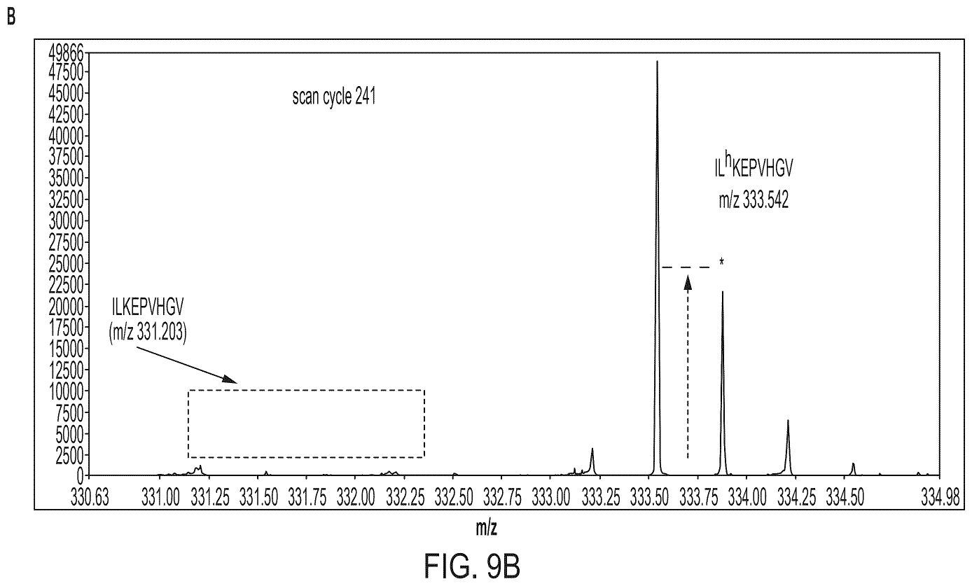

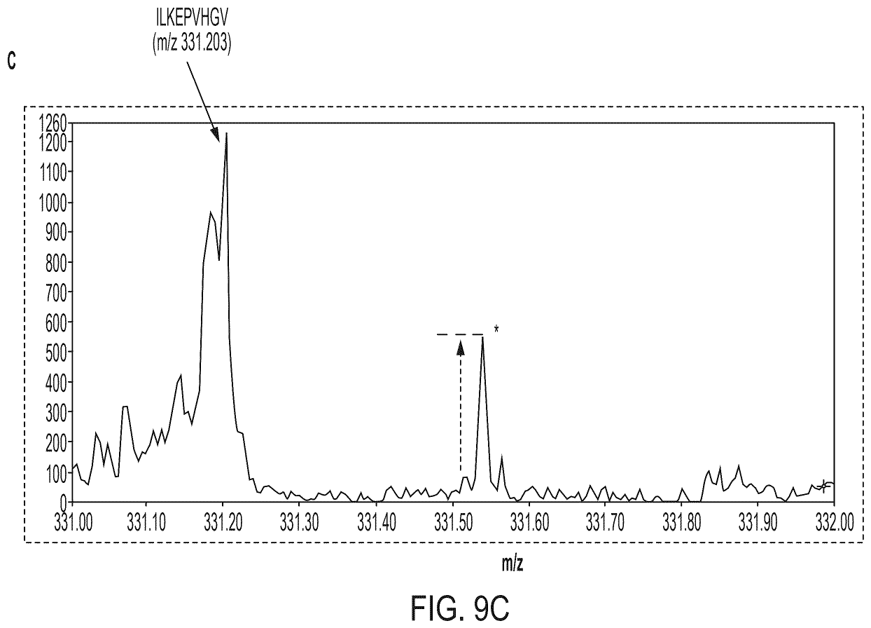

Poisson detection LC-DIAMS identified two epitopes physically present on the surface of GFP⁺ but not GFP⁻ cells: ALTEVVPLT (ALT) from reverse transcriptase (RT) and VLAEAMSQV (VLA) from the p2p7p1 region of the Gag polyprotein (FIGS. 1A-1E,Table 2, FIGS. 7A-7B). While ALT and VLA served as promising targets due to their physical identification on the surface of infected cells, we also analyzed as a positive control a previously characterized, highly conserved RT epitope ILKEPVHGV (ILK, Pol 464-472; RT 309-317), whose molecular ion we subsequently recognized in the 3^{rd} charged state at m/z 331.2 (Table 2). To quantitate the surface presentation of these peptides, we used targeted MS/MS Poisson detection with isotope labeled leucine (L*): AL*TEVVPLT, VL*AEAMSQV and IL*KEPVHGV. ALT was identified at 54 copies (FIGS. 1B, 8A, 8B) and VLA at 10 copies per cell (FIGS. 1C, 8A-8D). Surprisingly, although ILK has been identified as a target of CD8⁺T cells from PLWH, this peptide was detected from GFP⁺ cells at a copy number of <1/cell (FIGS. 1D-1E, 9A-9C). These results are particularly striking as the epitope derives from the same viral protein as ALT (FIG. 1F). Calculated pMHC copy numbers per cell are an average, and certain cells may express higher or lower amounts of a given pMHC. Even nominally higher levels of ILK (i.e. 1-5 copies) would be nearing the limit of CD8⁺T cell-based detection by the TCR (47, 48). Thus, we reasoned that the ILK pMHC-I target may provide insights into the limit of sensitivity of antibody-based retargeting strategies.

a Phage Display Library can be Used to Isolate Specific I-1 I-I scFvs.

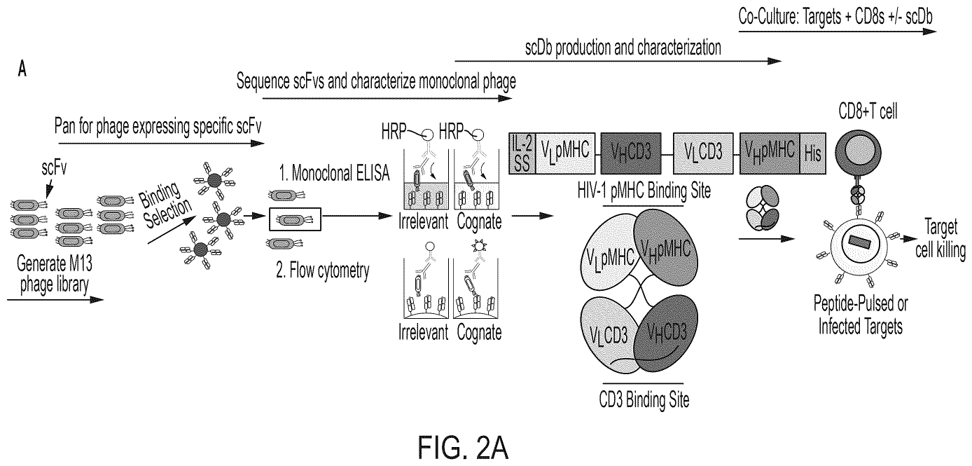

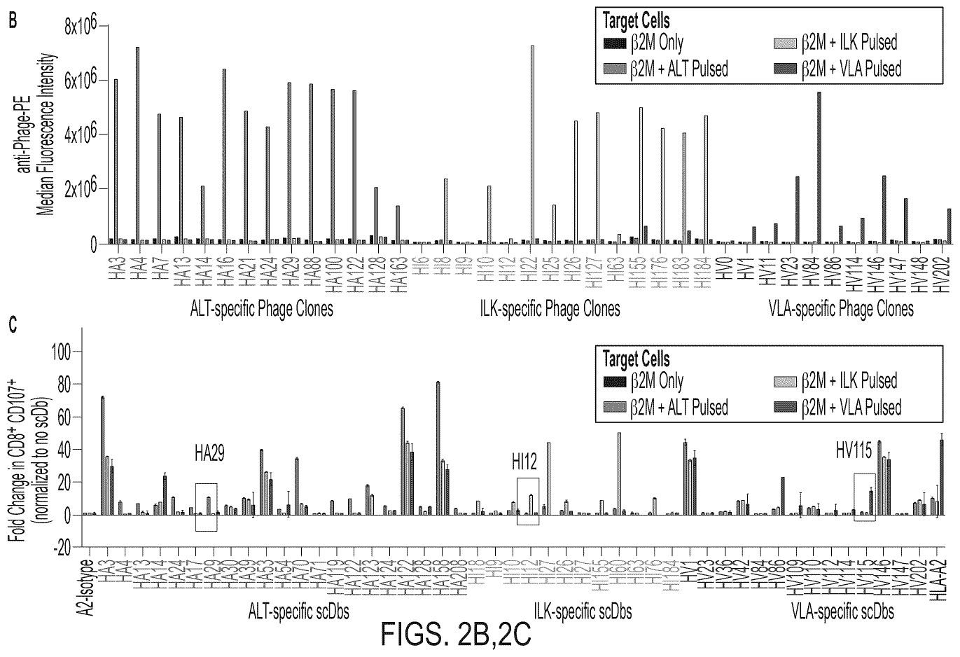

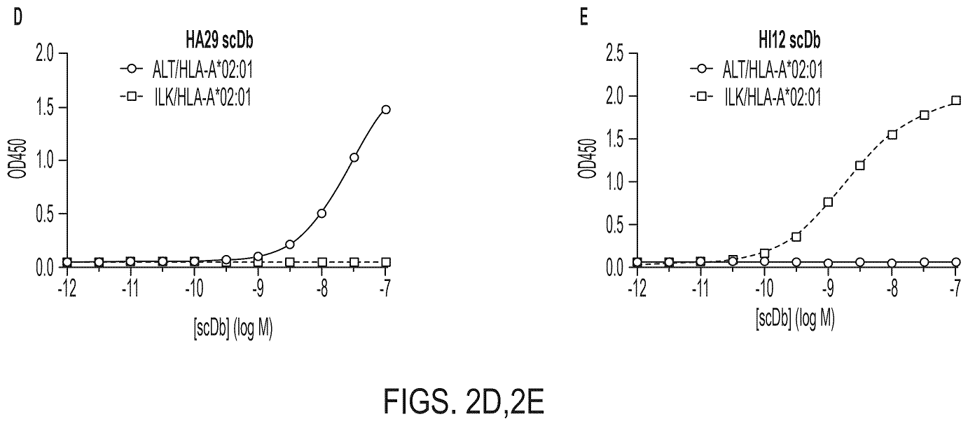

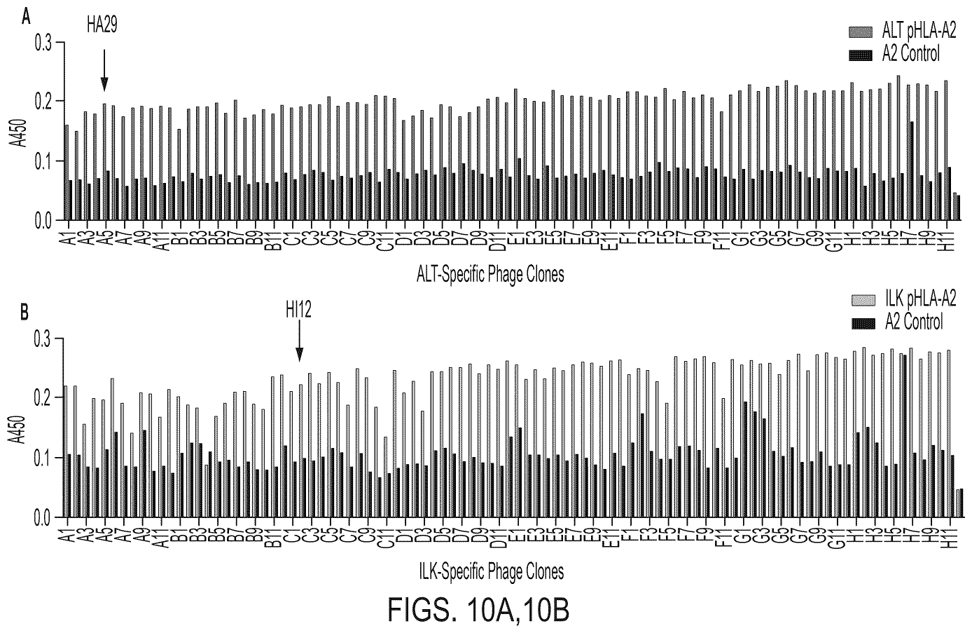

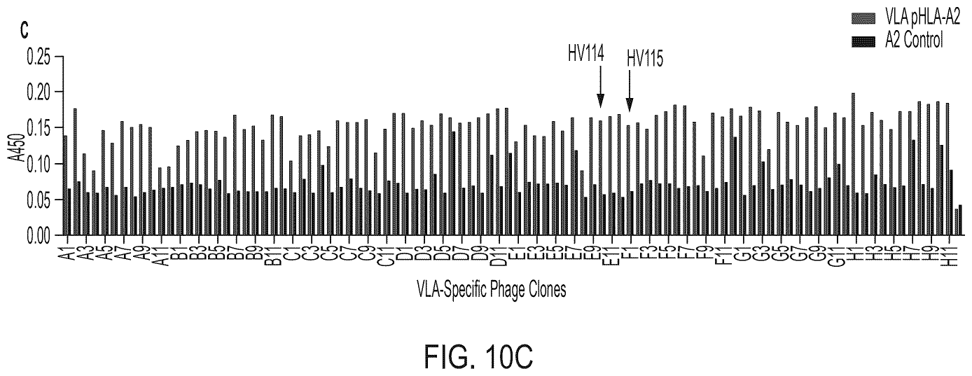

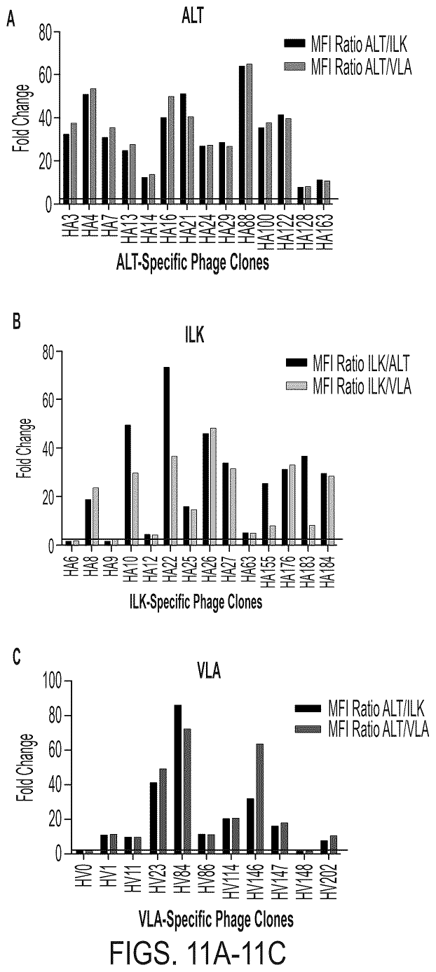

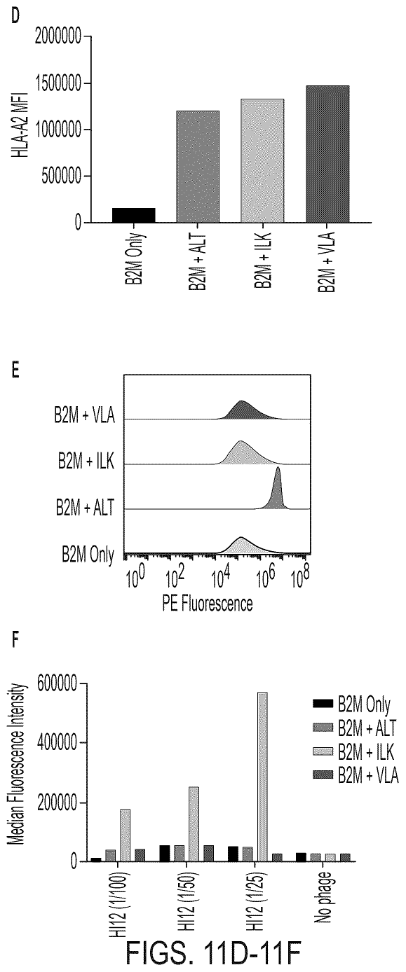

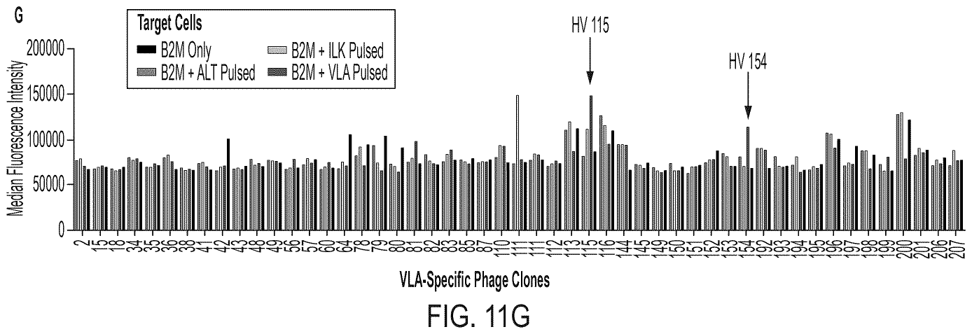

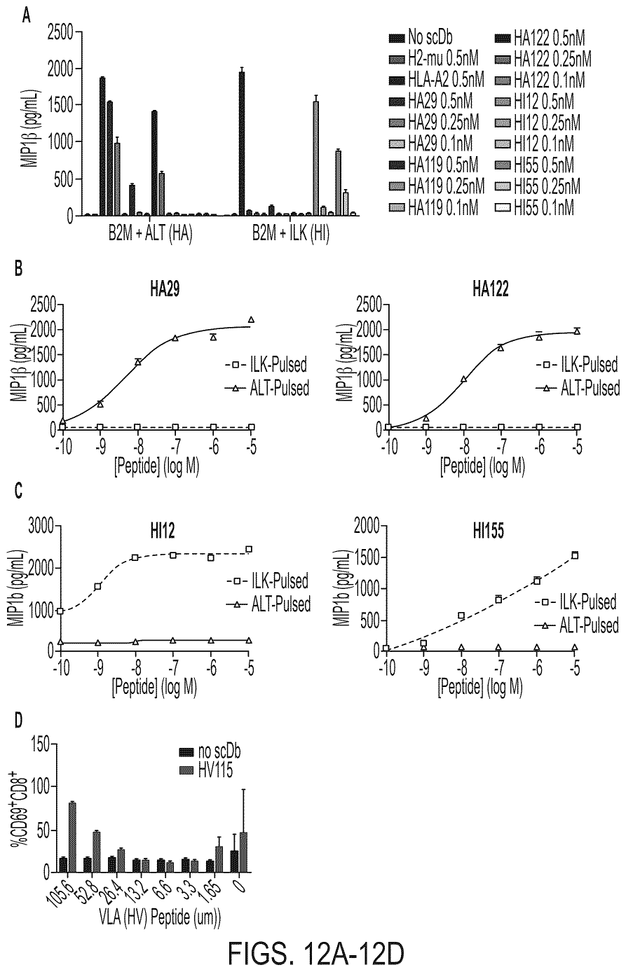

Given the difficulty of generating antibodies that recognize pMHC complexes, (49), we used a previously-generated phage display library with a diversity of ~3.6×10¹⁰ unique clones (50, 51) to screen for scFv-bearing phage that specifically bound HLA-A*02:01 in complex with ALT, ILK, and VLA peptides (FIG. 2A). Phage panning to enrich for scFv-bearing phage specific to these pMHC complexes involved positive selection against decreasing amounts of the cognate pMHC and negative selection against A2-expressing cell lines and streptavidin beads bound to biotinylated HLA-A2 complexes bearing irrelevant peptides (see Methods) (38, 39, 51). Monoclonal phage present after 4 rounds of panning were tested for specific binding to pMHC containing the cognate HIV-1 epitopes ALT (HA), ILK (HI), and VLA (HV), first by enzyme linked immunosorbent assay (ELISA) and then by flow cytometry. Phage scFv clones exhibiting >2-fold binding to the cognate epitope versus an irrelevant A2 pMHC on ELISA (FIGS. 10A-10C) were amplified. Selected phage by ELISA were then screened for binding to TAP-deficient T2 cells pulsed with cognate or irrelevant peptide epitopes, and phage binding was quantified by flow cytometry (FIGS. 2B, 11A-11C). All three peptide induced stabilization of HLA-A2 on T2 cells (FIG. 11D). We considered phage clones that bound to cognate versus irrelevant peptide-pulsed T2 cells at MFI ratios of >4 as “hits” and determined the scFv sequences of these clones (FIGS. 11A-11C, 11E). Phage “hits” at the lower limit of the threshold were assessed for enhanced binding with different amounts of phage (FIG. 11F). For the VLA/HLA-A*02:01 target, additional screening led to the identification of two specific phage clones, HV115 and HV154 (FIG. 11G). These clones exhibited the greatest specificity and binding to VLA/HLA-A*02:01, but exhibited a greater background binding than either HI or HA phage clones. In total, we identified 47 specific phage clones (HA=21, HI=12, HV=14).

HIV-1 pMHC-I scFvs can be Converted to Single-Chain Diabodies that Induce Specific T Cell Activation.

The scFv sequences of phage “hits” were converted into a single-chain diabody (scDb) format (38, 39) consisting of a single polypeptide chain containing the heavy and light chain variable regions of two scFv fragments separated by flexible glycine repeat linkers (FIG. 2A). The scFv of the anti-pMHC domain derived from phage panning flanks the scFv of the UCHT-1 clone of CD3e (38). Binding of the scDb to CD3 on the effector cell and the HIV-1 pMHC on the infected target cell tethers the effector to the target to form an immune cytolytic synapse, with activation of the effector cell as assessed by CD69 and CD107 upregulation, release of cytokines and chemokines such as IFNγ and MIP1β (38, 39), and target cell lysis.

To test the functionality of the scDbs, we Cocultures CD8⁺ T cells from healthy donors with T2 cells pulsed with cognate or irrelevant peptides and assessed effector cell activation by cell surface expression of CD107 (52) (FIG. 2C). Some scDbs were inactive or non-specific (FIG. 2C). The most specific phage clones against ALT were HA122 and HA29 (FIG. 2C). The most specific ILK clones were HI12 and HI55, and the most specific VLA clone was HV115 (FIG. 2C). HA29 and HI12 induced higher levels of CTL activation, as measured by secreted MIP1β, compared to HI55 and HA122 (FIG. S6A). These two scDbs also induced enhanced MIP1β production by and CD69 expression on CD8⁺T cells in response to cells pulsed with lower doses of cognate peptide compared with the other top scDbs HA122, HI55, or HV115 (FIG. S6B-D).

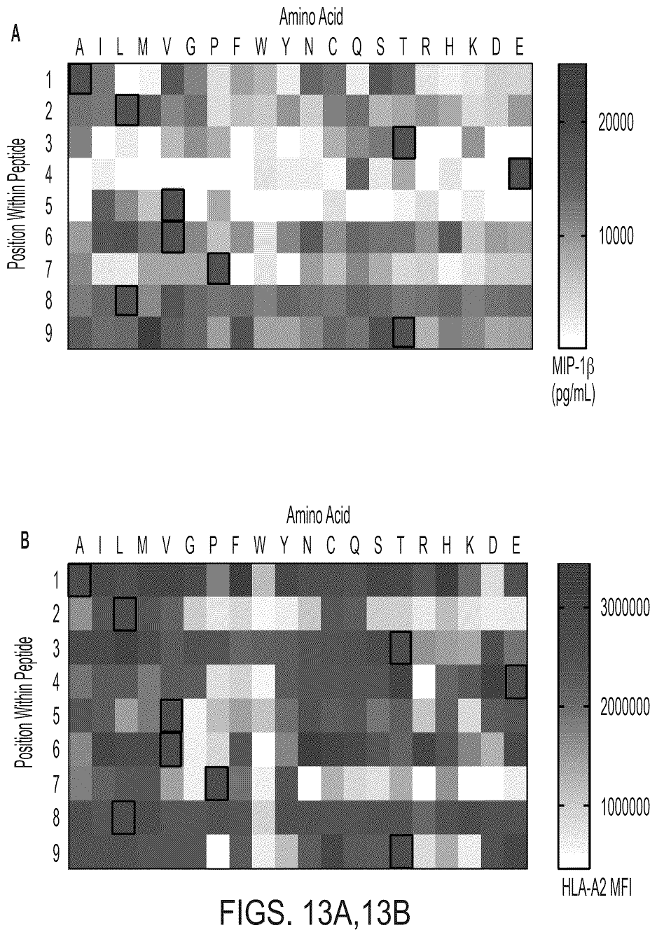

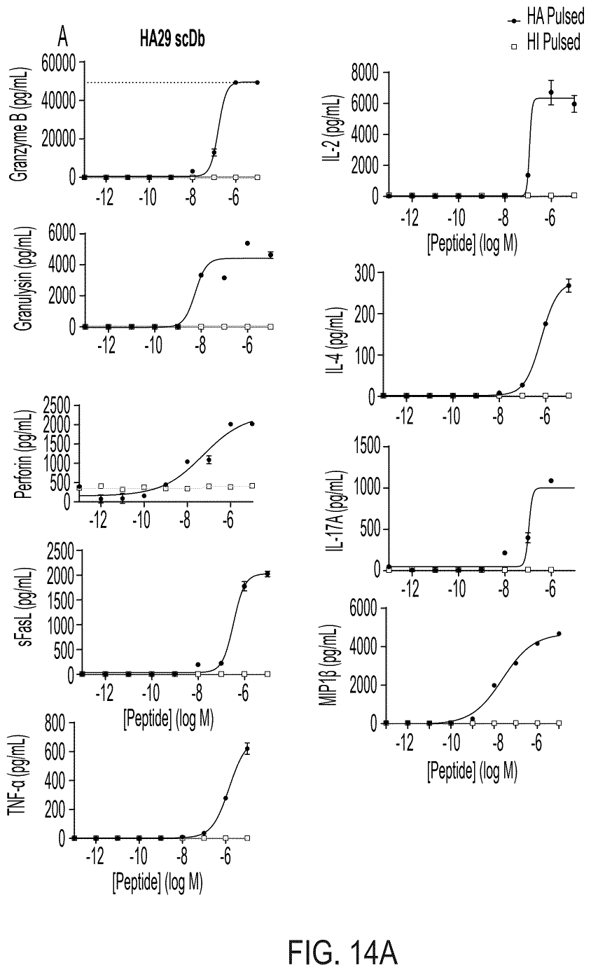

Additional testing of HA29 and HI12 scDbs using titration ELISAs highlighted their specificity. The HA29 scDb only bound to immobilized ALT/HLA-A*02:01 and not the irrelevant monomer ILK/HLA-A*02:01 even at high concentrations of HA29-scDb (FIG. 2D). Similarly, HI12 bound to only ILK/HLA-A*02:01 and not the irrelevant target ALT/HLA-A*02:01 (FIG. 2E). The specificity of HA29-scDb was additionally interrogated using positional scanning variant peptides, in which each residue of the ALT peptide was substituted with 19 other possible amino acids (38, 39). This resulted in a library of 171 variant peptides. HA29-induced upregulation of CTL activation was significantly decreased in co-cultures with T2 cells pulsed with peptide variants containing substitutions in positions 3-5 (FIGS. 13A-13B), suggesting that these residues were most critical for HA29 binding. Positions 1 and 7 tolerated more substitutions but also were critical for scDb binding (FIG. S7A-B), while substitutions at positions 6, 8, and 9 in many cases did significantly affect HA29 binding. Due to their superior activity and specificity, we pursued additional characterization of the scDbs HA29 and HI12.

HIV-Specific scDbs have High Affinity and Sensitivity for their Cognate pMHC.

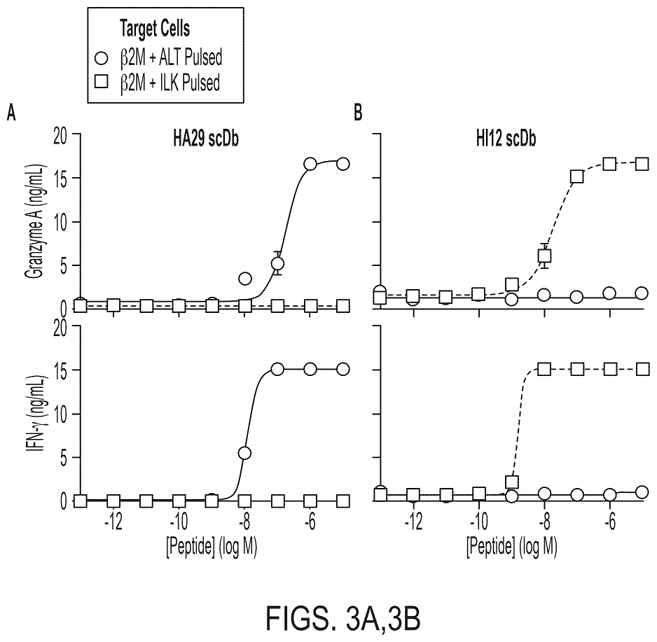

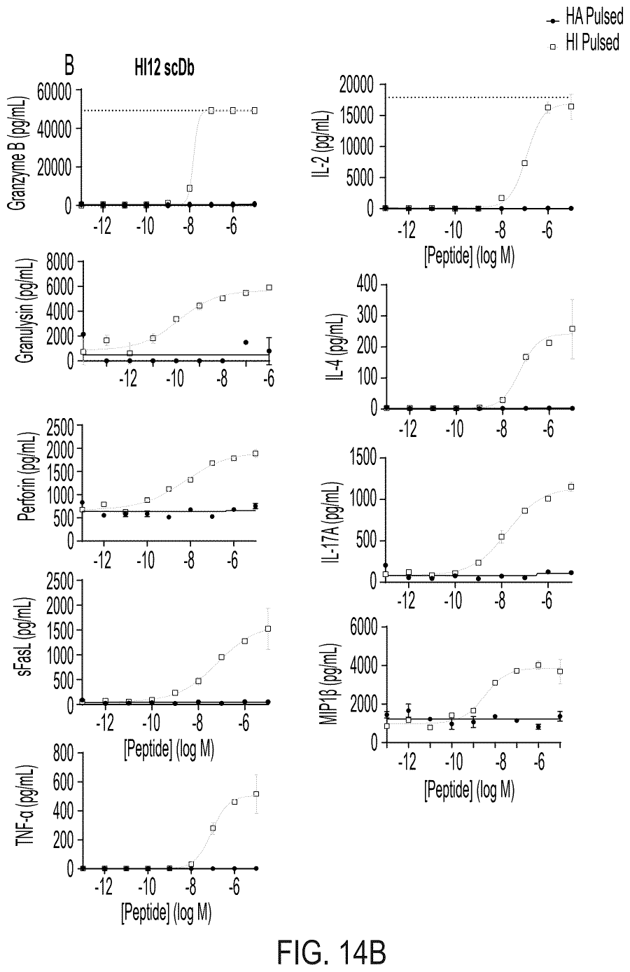

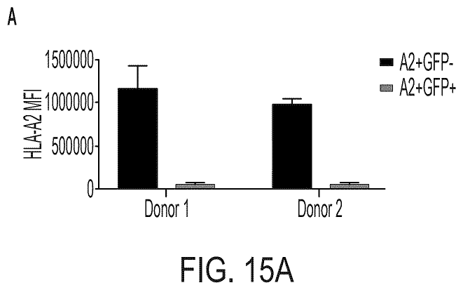

For TCRm reagents to promote killing of infected cells, they must detect viral peptides-MHC I complexes presented at low copies on target cells. We performed peptide titration co-cultures of T2 cells and healthy donor-derived CD8⁺T cells in the presence or absence of scDbs. At 0.25 nM, both HA29 and HI12 induced specific, polyfunctional T cell responses against cells pulsed with nanomolar concentrations of the relevant peptide (FIGS. 3A-3B; FIGS. 14A-14B). In addition to inducing canonical markers of CTL degranulation (granzyme A) and cytokine release (INF-7) (FIG. 3), we demonstrated specific induction of release of multiple additional granule proteins and effector molecules, cytokines, and chemokines including granzyme B, granulysin, perforin, sFasL, TNF-α, IL-2, IL-4, IL-17A, and MIP1β (FIGS. 14A, 14B).

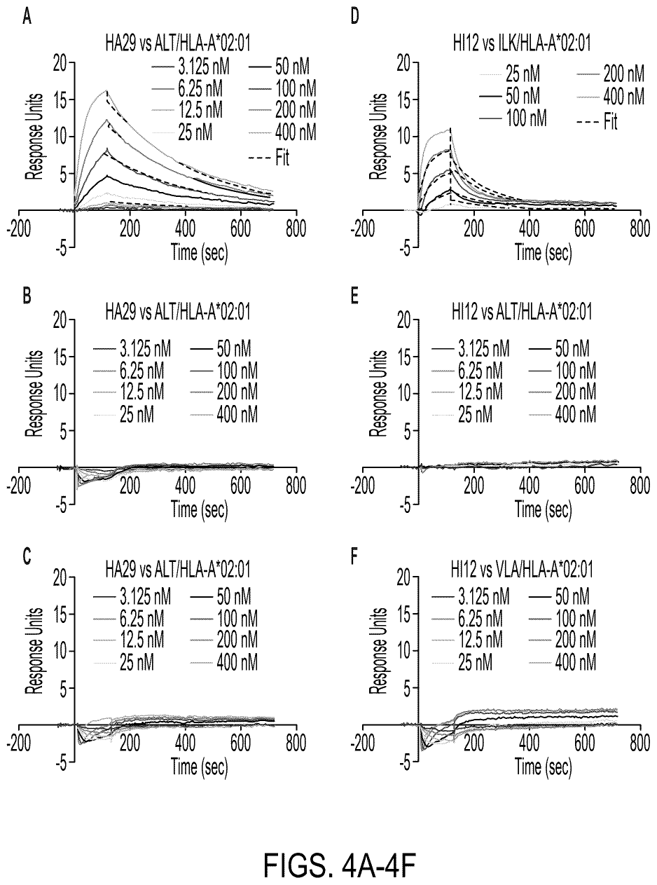

Surface plasmon resonance (SPR) analysis demonstrated HA29 binding to ALT/HLA-A*02:01 with an equilibrium constant (K_{D}) of 53.8 nM (FIG. 4A), with an association rate constant (kₒₙ) of 5.98×10⁴ M⁻¹s⁻¹ and a dissociation rate constant (k_{off}) of 3.22×10⁻³ M⁻¹s⁻¹. No binding of the HA29 scDb to ILK pHLA-A2 or VLA pHLA-A2 was detected (FIGS. 4B-4C). Similar analysis of HI12 demonstrated binding to ILK/HLA-A*02:01 with a K_{D} of 66.8 nM (FIG. 4D), a kₒₙ of 9.48×10⁴ M⁻¹s⁻¹, and a k_{off} of 6.58×10⁻³ M⁻¹s⁻¹. No binding of the HI12 scDb to ALT pHLA-A2 or VLA pHLA-A2 was detected (FIGS. 4E-4F). These data are consistent with the high potency and specificity of the HA29 and HI12 scDbs (FIGS. 3A-3B), suggesting that these scDbs may detect pMHC-I on infected cells and induce killing.

### HA29 and HI12 Induce Robust and Polyfunctional CTL Activation Against Peptide-Pulsed Cells.

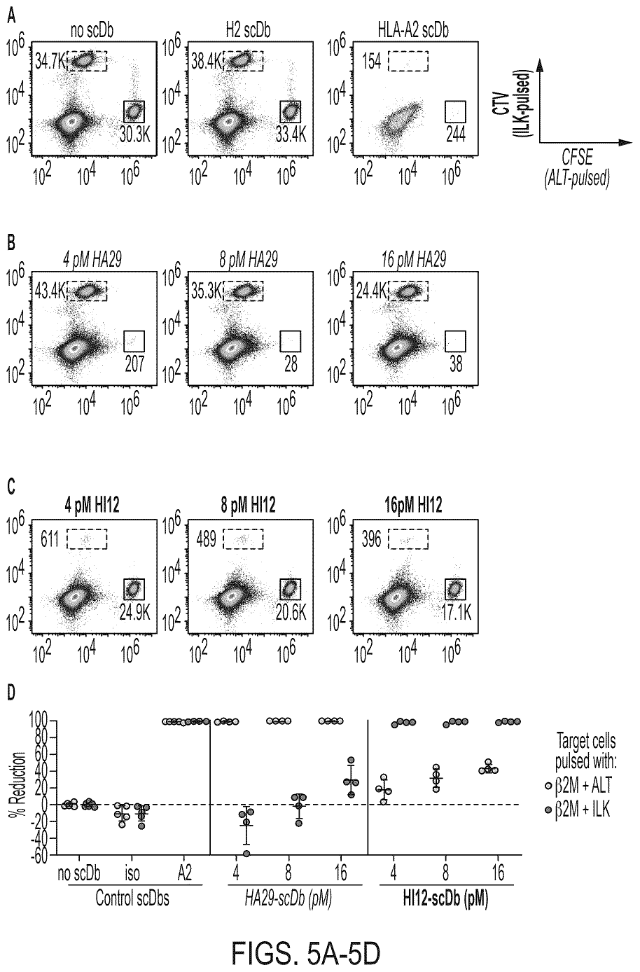

To test the ability of HA29 and HI12 to induce cytolysis of target cells bearing the relevant pMHC, T2 cells pulsed with the ALT or ILK peptides were labeled with CFSE or Cell-Trace Violet (CTV), respectively, and co-cultured with pre-activated CD8⁺T cells in the presence or absence of increasing doses of scDb. For negative controls, we used a no scDb condition (FIG. 5A, left) and an isotype scDb (H2) directed against a mutant p53 epitope presented on HLA-A*02:01 (FIG. 5A, middle) (39). We used a pan HLA-A2 (BB7.2) scDb as a positive control to identify the maximum killing of target populations (FIG. 5A, right). At picomolar concentrations, HA29 and HI12-scDbs induced nearly 100% reduction in t T2 cells pulsed with the relevant peptide (FIGS. 5B-5C), similar to the reductions obtained with 250 pM of the positive-control pan-A2 scDb (FIG. 5D). At the lowest concentrations of HA29 and HI12 (4 pM), there was minimal effect on targets pulsed with the irrelevant peptide.

Suppression of HIV-1 Using HA29 but not HI-12 scDbs.

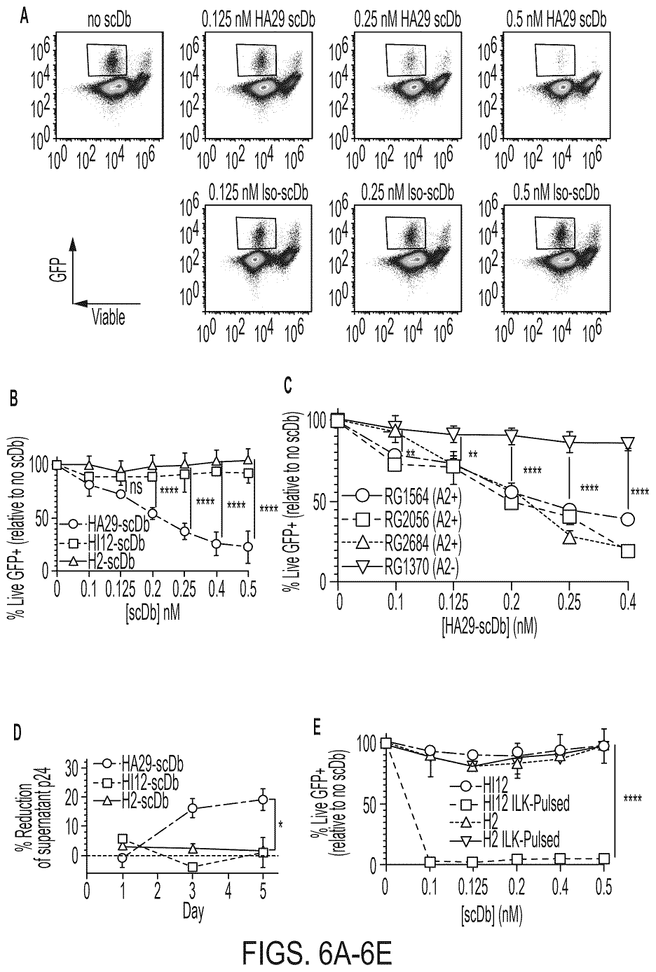

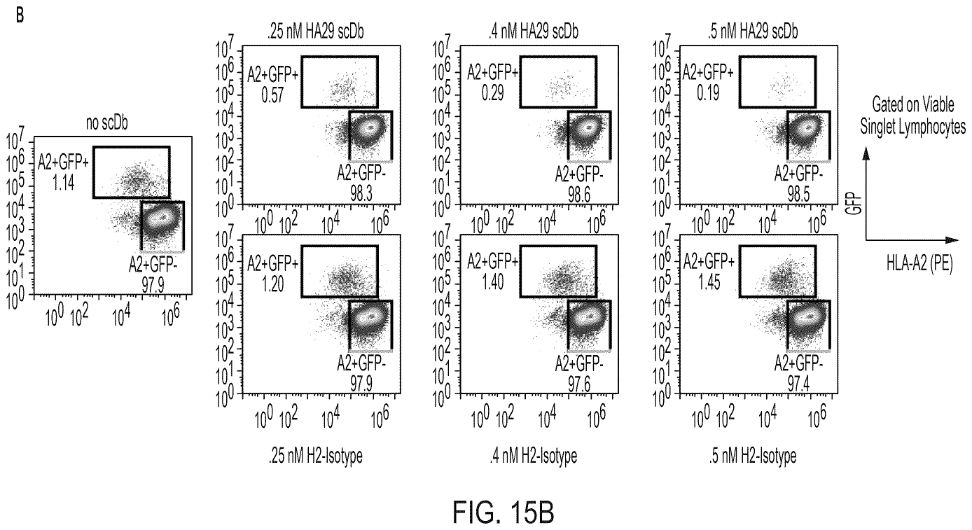

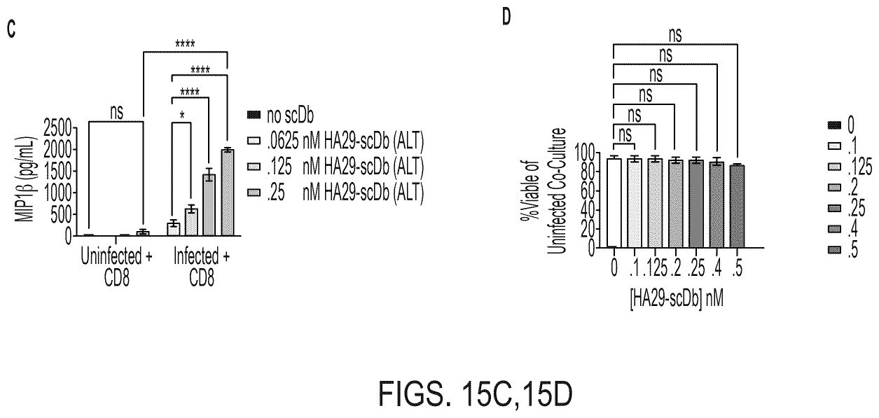

Given the HA29- and HI12-scDb induced killing of peptide-pulsed cells, we tested whether tscDbs could inhibit viral replication and/or induce direct lysis of infected cells using viral suppression assays (53). On infected cells, the level of specific peptide-MHC complexes is extremely low (54 copies of ALT/HLA-A*02:01/cell, FIGS. 8A-8B), too low to be detected by flow cytometry. Nevertheless, we hypothesized that the extremely sensitive nature of antigen recognition by T cells might allow scDb-directed lysis of infected cells. CD4⁺T cells from HLA-A2-expressing healthy donors were activated, infected with ΔEnv-NL4.3-EGFP (46), and co-cultured with autologous, pre-stimulated CD8⁺ T cells in the presence of HA29- or HI12-scDb at an E:T ratio of 3:1. We observed a striking dose-dependent decrease in viable GFP⁺ cells remaining after 3 days of co-culture with the HA29-scDb, but no decrease with the HI12 or irrelevant H2-scDb (FIGS. 6A-6B). At 0.4 nM HA29-scDb, the number of viable GFP⁺ cells was only 24% of that seen without scDb (FIG. 6B). These decreases reflected a dose-dependent increase in viral suppression, with the highest dose of HA29-scDb yielding 72% suppression of viral infection (FIG. 6C). The decrease in residual GFP⁺ cells mediated by HA29-scDb was dependent on HLA-A2, as no significant reductions in viable GFP⁺ cells were observed with an A2⁻ donor (FIG. 6D). HIV-1 Nef and Vpu downregulate MHC-I (54, 55), and we observed decreases in HLA-A2 on infected CD4⁺T cells (FIG. 15A). Despite lower A2 on infected cells, we observed suppression by HA29-scDb (FIG. 15B), suggesting that the residual numbers of viral pMHC-I were sufficient and the affinity of HA29-scDb was high enough to maintain a suppressive effect. Indeed, we observed HA29-mediated CTL activation even at low doses of the HA29-scDb in infected versus uninfected co-cultures (FIG. 15C). Importantly, we did not observe a significant decrease in viability in uninfected CD4⁺T cells co-cultured with autologous CD8⁺ T cells in the presence of HA29-scDb (FIG. 15D).

Having shown viral suppression using a standard HIV-1 reporter virus, we next confirmed that HA29-scDb could suppress a replication-competent primary isolate from the latent reservoir of a treated PLWH. We infected primary CD4 T cells with isolate 33A10 (56) and measured p24 production over time with and without scDbs in the presence of autologous CD8⁺ T cells. Levels of p24 antigen were reduced by the HA29-scDb (FIG. 6D). Together, these results demonstrate that the HA29-scDb can induce CD8⁺ T cell-mediated killing of HIV-1-infected cells. This is dependent on the presence of A2 and antigen. Interestingly, as with ΔEnv-NL4.3-EGFP-infected cells (FIG. 6B) we did not observe a suppressive effect with the HI12 scDb, despite its high sensitivity for detecting T2 cells pulsed with nanomolar concentrations of peptide (FIGS. 3B, 6B, 14B) and its high affinity for ILK/HLA-A*02:01 (FIGS. 4D-4F). The inability of the HI12-scDb to mediate killing may be due to lower levels of ILK pMHC-I on infected cells, as suggested by quantification of ILK peptide from GFP⁺ cells by MS (FIGS. 9A-9B). Indeed, when we pulsed infected CD4⁺T cells with saturating a concentration (10 ug/ml) of the ILK peptide, we observed a nearly 100% decrease in the residual viable GFP⁺ population compared to the irrelevant H2-scDb (FIG. 6F). Therefore, antigen density on HIV-1-infected cells may limit the effectiveness even of even high affinity scDbs.

TABLE 1
Predicted HLA-A*02:01 binding peptides
from the HIV-1 proteome (excluding Env) for
analysis by LC-DIAMS. (SEQ ID NOs: 1-107)

| | | Amino Acid |
|---|---|---|
| Sequence* | Protein | Position† |
| ILGQLQPSL | Gag | 60 → 68 |
| SLQTGSEEL | Gag | 67 → 75 |
| RSLYNTIAVL | Gag | 76 → 85 |
| SLYNTIAVL | Gag | 77 → 85 |
| [symbol] | Gag | 79 → 88 |
| [symbol] | Gag | 80 → 88 |
| [symbol] | Gag | 84 → 92 |
| SQVSQNYPI | Gag | 126 → 134 |
| TLNAWVKVV | Gag | 151 → 159 |
| QDLNTMLNTV | Gag | 182 → 191 |
| [symbol] | Gag | 209 → 218 |
| MTHNPPIPV | Gag | 250 → 258 |
| [symbol] | Gag | 267 → 276 |
| RMYSPTSIL | Gag | 275 → 283 |
| ATLEEMMTA | Gag | 341 → 349 |
| [symbol] | Gag | 345 → 353 |
| RVLAEAMSQV | Gag | 361 → 370 |
| [symbol] | Gag | 362 → 370 |
| VLAEAMSQVT | Gag | 362 → 371 |
| AMSQVTNPA | Gag | 366 → 374 |
| FLQSRPEPT | Gag | 448 → 456 |
| FLQSRPEPTA | Gag | 448 → 457 |
| DILDLWIYHT | Nef | 108 → 117 |
| ILDLWIYHT | Nef | 109 → 117 |
| [symbol] | Nef | 137 → 145 |
| [symbol] | Nef | 137 → 146 |
| [symbol] | Nef | 139 → 148 |
| [symbol] | Nef | 144 → 153 |
| GMDDPEREV | Nef | 172 → 180 |
| KMIGGIGGFI | Pol | 101 → 110 |
| [symbol] | Pol | 113 → 122 |
| [symbol] | Pol | 118 → 127 |
| TVLVGPTPV | Pol | 130 → 138 |
| [symbol] | Pol | 144 → 153 |
| TLNFPISPI | Pol | 152 → 160 |
| [symbol] | Pol | 187 → 196 |
| [symbol] | Pol | 188 → 196 |
| VQLGIPHPA | Pol | 245 → 253 |
| YTAFTIPSI | Pol | 282 → 290 |
| [symbol] | Pol | 313 → 322 |
| [symbol] | Pol | 335 → 344 |
| YMDDLYVGS | Pol | 338 → 346 |
| HLLRWGFTT | Pol | 363 → 371 |
| FLWMGYELHP | Pol | 382 → 391 |
| [symbol] | Pol | 387 → 396 |
| VLPEKDSWTV | Pol | 400 → 409 |
| KLVGKLNWA | Pol | 414 → 422 |
| [symbol] | Pol | 436 → 444 |
| KALTEVVPL | Pol | 442 → 450 |
| KALTEVVPLT | Pol | 442 → 451 |
| [symbol] | Pol | 443 → 451 |
| [symbol] | Pol | 464 → 472 |
| [symbol] | Pol | 563 → 572 |
| YQLEKEPII | Pol | 582 → 590 |
| PIIGAETFYV | Pol | 588 → 597 |
| IIGAETFYV | Pol | 589 → 597 |
| IIGAETFYVD | Pol | 589 → 598 |
| LALQDSGLEV | Pol | 639 → 648 |
| ALQDSGLEV | Pol | 640 → 648 |
| IVTDSQYAL | Pol | 650 → 658 |
| VLFLDGIDKA | Pol | 714 → 723 |
| RAMASDFNL | Pol | 735 → 743 |
| MASDFNLPPV | Pol | 737 → 746 |
| ASDFNLPPV | Pol | 738 → 746 |
| [symbol] | Pol | 767 → 775 |
| [symbol] | Pol | 782 → 790 |
| [symbol] | Pol | 782 → 790 |
| KVILVAVHV | Pol | 786 → 794 |
| LLDTGADDTV | Pol | 79 → 88 |
| YIEAEVIPA | Pol | 798 → 806 |
| [symbol] | Pol | 816 → 825 |
| [symbol] | Pol | 886 → 895 |
| KLLWKGEGA | Pol | 955 → 963 |
| KLLWKGEGAV | Pol | 955 → 964 |
| LLWKGEGAV | Pol | 956 → 964 |
| LLWKGEGAVV | Pol | 956 → 965 |
| [symbol] | Pol | 988 → 996 |
| TYLGRSAEPV | Rev | 62 → 71 |
| YLGRSAEPV | Rev | 63 → 71 |
| QILVESPTV | Rev | 101 → 109 |
| ILVESPTVL | Rev | 102 → 110 |
| [symbol] | Vif | 5 → 13 |
| [symbol] | Vif | 22 → 31 |
| KISSEVHIPL | Vif | 50 → 59 |
| LVITTYWGL | Vif | 64 → 72 |
| SLQYLALAA | Vif | 144 → 152 |
| SLQYLALAAL | Vif | 144 → 153 |
| LQYLALAAL | Vif | 145 → 153 |
| KQIKPPLPSV | Vif | 157 → 166 |
| [symbol] | Vpr | 18 → 26 |
| LLEELKSEAV | Vpr | 22 → 31 |
| [symbol] | Vpr | 61 → 70 |
| RILQQLLFI | Vpr | 62 → 70 |
| [symbol] | Vpr | 66 → 74 |
| [symbol] | Vpu | 3 → 13 |
| [symbol] | Vpu | 5 → 13 |
| [symbol] | Vpu | 5 → 14 |
| AIVALVVAI | Vpu | 8 → 16 |
| [symbol] | Vpu | 11 → 20 |
| [symbol] | Vpu | 12 → 20 |
| [symbol] | Vpu | 12 → 21 |
| [symbol] | Vpu | 13 → 21 |
| [symbol] | Vpu | 16 → 25 |
| [symbol] | Vpu | 17 → 25 |
| [symbol] | Vpu | 17 → 26 |
| [symbol] | Vpu | 41 → 50 |
| ALVEMGVEM | Vpu | 63 → 71 |

*No shading indicates peptides with fragmentation patterns detected by Poisson LC-DIAMS (64 peptides). Peach shading indicates peptides for which fragmentation patterns could not be identified by LC-DIAMS (43 peptides). Green shading indicates peptides that yielded fragmentation patterns by LC-DIAMS and were detected on infected (GFP+) cells (3 peptides).
*Numbers represent amino acid position relative to protein start in HXB2.

TABLE 2
Characteristics of each pMHC-I target

| scDb | HLA | Epitope | Epitope |
|---|---|---|---|
| Identifier | Haplotype | Specificity | Location |
| HA | A*02:01 | ALTEVVPLT | Pol (RT) |
| HI | A*02:01 | ILKEPVHGV | Pol (RT) |
| HV | A*02:01 | VLAEAMSQV | Gag (p24-p2) |

TABLE 3
Sequences of V_{H} and V_{L} for relevant scDbs

| scFv | VH | VL |
|---|---|---|
| HV115-scDb | EVQLVESGGGLVQPGGSLRLSCAA | DIQMTQSPSSLSASVGDRVTITCRASQD |
| | SGFNVTSVQMHWVRQAPGKGLEWV | VNTAVAWYQQKPGKAPKLLIYSASFLYS |
| | AMFYPDSDYTMYADSVKGRFTISA | GVPSRFSGSRSGTDFTLTISSLQPEDFA |
| | DTSKNTAYLQMNSLRAEDTAVYYC | TYYCQQYYYWYPITFGQGTKVEIKRT |
| | SRMSYSSAFDYWGQGTLVTVSS | SEQ ID NO: 109 |
| | SEQ ID NO: 108 | |
| HI12-scDb | DIQMTQSPSSLSASVGDRVTITCR | EVQLVESGGGLVQPGGSLRLSCAASGFN |
| | ASQDVNTAVAWYQQKPGKAPKLLI | ISGGSMHWVRQAPGKGLEWVAYVYPQSG |
| | YSASFLYSGVPSRFSGSRSGTDFT | NTYYADSVKGRFTISADTSKNTAYLQMN |
| | LTISSLQPEDFATYYCQQWDYHYS | SLRAEDTAVYYCSRYYIYGLDVWGQGTL |
| | PVTFGQGTKVEIK | VTVSS |
| | SEQ ID NO: 110 | SEQ ID NO: 111 |
| HA29-scDb | DIQMTQSPSSLSASVGDRVTITCR | EVQLVESGGGLVQPGGSLRLSCAASGFN |
| | ASQDVNTAVAWYQQKPGKAPKLLI | FSWSSIHWVRQAPGKGLEWVAQLSYYSD |
| | YSASFLYSGVPSRFSGSRSGTDFT | YTNYADSVKGRFTISADTSKNTAYLQMN |
| | LTISSLQPEDFATYYCQQYYSSPV | SLRAEDTAVYYCSRGPYYMDYWGQGTLV |
| | TFGQGTKVEIK | TVSS |
| | SEQ ID NO: 112 | SEQ ID NO: 113 |
| H2-scDb | DIQMTQSPSSLSASVGDRVTITCR | EVQLVESGGGLVQPGGSLRLSCAASGFN |
| (39) | ASQDVNTAVAWYQQKPGKAPKLLI | VYASGMHWVRQAPGKGLEWVAKIYPDSD |
| | YSAYFLYSGVPSRFSGSRSGTDFT | YTYYADSVKGRFTISADTSKNTAYLQMN |
| | LTISSLQPEDFATYYCQQYSRYSP | SLRAEDTAVYYCSRDSSFYYVYAMDYWG |
| | VTFGQGTKVEIK | QGTLVTVSS |
| | SEQ ID NO: 114 | SEQ ID NO: 115 |

### DISCUSSION

The importance of a robust CD8⁻T cell response in HIV-1 infection is highlighted by studies of elite suppressors (ES), who suppress viremia to undetectable levels without cART (57, 58). The magnitude and characteristics of the CD8⁺ T cell response in ES (19, 59, 60), and the targeting of highly networked epitopes (61), have been implicated in their ability to control viral replication. These studies have led to efforts to stimulate CTLs in PLWH using therapeutic vaccination strategies for cure efforts. This has proven challenging, however, as HIV-1-specific CTLs in chronic progressors have a hypofunctional phenotype that is not completely restored by cART (18, 19, 62, 63). HIV-1-specific CTLs also require pre-stimulation in order to clear infected cells after latency reversal (20). Re-targeting strategies, such as the pMHC-T scDb strategy outlined above, are beneficial in that they enable CD8⁺ T cells of any specificity to recognize HIV-infected cells, thereby bypassing defects unique to the HIV-1-specific CTL subpopulation.

Here we describe bispecific antibodies against HIV-1 pMHC-I expressed on infected cells. Bispecific antibodies capable of detecting rare HIV-1 pMHC complexes on infected cells might be useful in eradicating the latent reservoir following latency reversal. To identify target peptides, we used a highly sensitive mass spectrometry method to quantitate HIV-1 peptides presented on MHC-I isolated from infected cells. Phage bearing scFv specific for the identified pMHC-T were then isolated by panning from an extremely diverse library and cloned into a single-chain diabody backbone.

Two scDbs, HA29 and HI12, exhibited high affinity and specificity for their respective pMHC targets. Subnanomolar concentrations of both scDbs induced polyfunctional CTL activation against T2 cells pulsed with peptides in the 0.1-10 nM range. Prior studies involving single-molecule immunofluorescence using an affinity-matured TCR against a melanoma pMHC suggest that peptide concentrations of 0.1-10 nM should result in the presentation of physiological levels of pMHC (4-80 copies per cell) (64). Since the HA29 and HI12 bispecific antibodies enabled CD8⁺ T cell recognition of cells pulsed with 0.1 nM peptide, we predicted these antibodies detect endogenously processed epitopes on infected cells. When HA29 and HI12-scDbs were incubated with CTLs and newly infected CD4⁺ T cells, HA29-scDb suppressed viral infection in a 72 hr co-culture. In contrast, the HI12-scDb did not, consistent with the lower copy number of the ILK epitope recognized by HI12 detected as quantitated by mass spectrometry (<1 copy of ILK/HLA-A*02:01 per cell). Pulsing infected cells with the ILK peptide and then co-culturing them with CTLs led to dramatic suppression/killing, implicating low antigen density as the primary factor preventing HI12-induced killing.

Our results are consistent with prior studies. Tsomides et al. generated a stable, HLA-A2⁺ Jurkat cell line carrying a nef-deficient HIV-1 provirus and estimated the ILK pMHC density at ~10 copies/cell (65). An ILK-specific T cell clone required very high (i.e. 50:1) E:T ratios to promote killing of the cell line (65). If the density of ILK on cells constitutively expressing HIV-1 protein and A2 levels is only ~10 copies (66), it follows that in the setting of natural infection, presentation of ILK on the cell surface would be even lower. ILK is derived from the RT protein, which is expressed at only 10% of the level of the more abundant Gag protein due to a ribosomal frame shift required for RT translation, but this alone is not sufficient to explain the low levels of ILK. The ALT peptide, found at 54 copies per cell, derives from the same protein. Thus, the difference between the RT ALT and ILK copy numbers (54 vs <1/cell) may reflect differences in processing. Although two epitopes are located within 12 residues of each other, ALT (aa 290-298) is located on an extruded loop of RT and contains 1 residue of an a-helix that precedes ILK (aa 309-317). ILK is located at the C-terminal end of the same constrained a-helix that separates these two epitopes. The entire 17 amino acid helix is likely less easily unfolded and, thus, may impact translocation through the proteosome and/or protein cleavage to diminish the generation of the downstream ILK epitope (67). Importantly, other factors may also contribute to the efficacy of the scDb approach describe here. In addition to peptide affinity for the relevant MHC molecule, these include the effect of flanking residues on cleavage by proteasome and also other amino peptidases such as ERAP, TAP transport, association with molecular chaperones, and stability of the complexes.

Our results highlight the need to re-examine the optimal CTL epitopes for viral control in the context of antigen density (68). Most studies have conservation of CTL epitopes (21, 69, 70). Recently, network analysis has identified epitopes that are mutationally constrained (61). The consensus is that targeting highly conserved or mutationally constrained epitopes would be therapeutically beneficial. However, highly conserved epitopes such as ILK may not be presented at sufficient levels to serve as appropriate targets. Responses to the ILK epitope were first observed using T cell clones from asymptomatic HIV⁻ individuals that lysed autologous cells infected with vaccinia vectors expressing HIV-1 genes (71, 72). However, vaccinia infection may produce proportionally more viral pMHC than would occur from natural HIV-1 infection (65).

Many cure strategies target HIV-1 Env on the surface of infected cells (73). However, Env has an extremely high sequence diversity (74). Broadly-neutralizing antibodies (bNAbs) address this issue (75) and have been reformatted to include anti-CD3 domains to engage T cells (33, 34). However, anti-Env bNAbs can drive selective pressure (76) and the cell-surface levels of HIV-1 Env are low on infected cells (36, 37). Anti-pMHC scDbs could synergize with anti-Env scDbs to enhance CTL killing of infected cells following latency-reversal, providing a complementary arm for the “kill” in shock and kill efforts.

There are several potential benefits to using pMHC-specific scDbs. The large diversity of targets against which scDbs can be generated throughout the viral proteome provides a level of versatility that anti-Env bNAbs do not offer. A “cocktail” of bispecific engagers—targeting conserved pMHC presented at relatively high copy numbers from various viral proteins—could trigger CTL-mediated lysis of infected cells in a manner that would preclude viral evasion from any one scDb. Although the scDbs described here showed high affinity and specificity for the target pMHC, higher concentrations of the scDbs exhibited some non-specific activity against irrelevant targets. Higher affinity and specificity could be achieved by additional scFv selection using affinity-matured libraries. The same methodology can be extended to other HLA alleles to increase the population coverage. SIV antigens in infected cells may be processed and presented using alternative pathways on MHC-II and HLA-E (77-79). As HLA-E is nearly monomorphic, targeting a conserved HIV-1 peptide bound to HLA-E using the above approach may provide a nearly universal bispecific reagent. Thus, the strategies described here could be used to isolate scFv specific for non-classically presented HIV-1 peptides. Another benefit of scDb is that they induce CTL activation even at low antigen densities, as seen from our peptide-pulsing experiments.

Whether scDb are able to activate CTLs better than natural recognition by αβTCRs remains to be seen. In this regard, very low pMHC densities may cause an anergic phenotype in certain effector CTLs encountered in vivo, which could limit effectiveness (80). Contrary to conventional receptor-ligand interactions exemplified by antigen-antibody binding, bioforces are essential for non-thermal equilibrium, mechanosensor-based αβT cell activation (81-89). αβT cell motility during immune surveillance and the local cytoskeletal machinery place physical load on individual αβTCR-pMHC bonds, tuning the sensitivity and specificity of αβTCR recognition (81). In the absence of external load, chemical thresholds to trigger cellular αβT cell activation require a thousand fold or higher number of pMHC molecules than observed physiologically (82). In contrast, under force the ligand-mediated induction of αβT cell biological response can be essentially digital, i.e. single pMHC recognition. That the, it remains to be seen how soluble bispecific reagents versus digitally performing αβTCRs used for adoptive cellular immunotherapy compare head-to-head in detection of the same pMHC ligand on target cells to effectuate specific killing. Clustering of bispecific reagents, perhaps on nanoparticle arrays, could potentially increase sensitivity.

These scDbs will promote the elimination of latently infected cells if HIV-1 gene expression when combined with induction by effective LRAs. The discovery of LRAs that induce viral gene expressing in a large fraction of reservoir cells is a problem that has not yet been solved despite extensive effort in many laboratories (12, 14-18, 90). The discovery of more effective LRAs that will complement the killing strategy described here. In addition, the persistence of HIV-1 in macrophages and in T cells subsets that are localized to tissues sites not readily penetrated by CTL (such as germinal centers) may pose additional barriers to the elimination of infected cells (92,93). Furthermore, reservoir cells may have been selected for intrinsic resistance to CTL killing (94). It is also important to note that many reservoir viruses contain escape mutations in immunodominant CTL epitopes (21). Fortunately, mutations are generally absent in subdominant epitopes, allowing targeting of infected cells (21). Finally, CTL dysfunction associated with the expression of PD1 may persist in the setting of ART (95). However, the exhausted phenotype is found on HIV-1-specific CTL and to a much less extent on CTL specific for other antigens. An advantage of the strategy described here is that it enables CTLs of any specificity to kill HIV-1-infected target cells.

The complications described above may contribute to the finding that “shock and kill” strategies have yet to produce substantial decrease in the latent reservoir in animal models (90). However, there is evidence for a delay in viral rebound after ART interruption in SIV-infected macaques that received both an immune stimulant with potential LRA activity (TLR7 agonist) coupled with immunologic interventions (broadly neutralizing antibodies or therapeutic vaccination) (96,97). The small signal observed in these studies provides hope that cure may be possible with better LRAs and more effective killing strategies, possibly including the one described here.

To our knowledge, no other studies have described the generation and use of TCRm-antibodies against HIV pMHC to target latent HIV infection. We have identified naturally-processed targets on infected cells and generated reagents using phage display that could recognize and kill infected cells. Our studies highlight the importance of antigen density in the design of therapeutic vaccines and immunotherapies and lay the groundwork for future studies that aim to eliminate the latent reservoir using bispecific cell engagers.

### REFERENCES

1. T. W. Chun, et al., In vivo fate of HIV-1-infected T cells: Quantitative analysis of the transition to stable latency. Nat. Med. 1, 1284-1290 (1995).
2. Chun et al., Quantification of latent tissue reservoirs and total body viral load in HIV-1 infection. Nature 387, 183-188 (1997).
3. T. W. Chun, K. Chadwick, J. Margolick, R. F. Siciliano, Differential susceptibility of naive and memory CD4+ T cells to the cytophatic effects of infection with human immunodeficiency virus type 1 strain LAI. J Virol 71, 4436-4444 (1997).
4. D. Finzi, et al., Identification of a Reservoir for HIV-1 in Patients on Highly Active Antiretroviral Therapy Identification of a Reservoir for HIV-1 in Patients on Highly Active Antiretroviral Therapy. Science. 278, 1295-1300 (1997).
5. J. K. Wong, et al., Recovery of replication-competent HIV despite prolonged suppression of plasma viremia. Science. 278, 1291-1295 (1997).
6. R. T. Davey, et al., HIV-1 and T cell dynamics after interruption of highly active antiretroviral therapy (HAART) in patients with a history of sustained viral suppression. Proc. Natl. Acad. Sci. U.S.A. 96, 15109-14 (1999).
7. Chun et al., Re-emergence of HIV after stopping therapy. Nature 401, 874-875 (1999).
8. D. Finzi, et al., Latent infection of CD4+ T cells provides a mechanism for lifelong persistence of HIV-1, even in patients on effective combination therapy. Nat. Med. 5, 512-517 (1999).
9. J. D. Siliciano, et al., Long-term follow-up studies confirm the stability of the latent reservoir for HIV-1 in resting CD4+ T cells. Nat. Med. 9, 727-728 (2003).
10. M. C. Strain, et al., Heterogeneous clearance rates of long-lived lymphocytes infected with HIV: Intrinsic stability predicts lifelong persistence. PNAS 100, 4819-24 (2003).
11. A. M. Crooks, et al., Precise Quantitation of the Latent HIV-1 Reservoir: Implications for Eradication Strategies. J. Infect. Dis. 212, 1361-1365 (2015).
12. N. M. Archin, J. M. Sung, C. Garrido, N. Soriano-Sarabia, D. M. Margolis, Eradicating HIV-1 infection: Seeking to clear a persistent pathogen. Nat. Rev. Microbiol. 12, 750-764 (2014).
13. A. R. M. Townsend, et al., The epitopes of influenza nucleoprotein recognized by cytotoxic T lymphocytes can be defined with short synthetic peptides. Cell 44, 959-68 (1986).
14. N. M. Archin, et al., Administration of vorinostat disrupts HIV-1 latency in patients on antiretroviral therapy. Nature 487, 482-5 (2012).
15. J. H. Elliott, et al., Activation of HIV Transcription with Short-Course Vorinostat in HIV-Infected Patients on Suppressive Antiretroviral Therapy. PLoS Pathog. 10, e1004473. (2014).
16. T. A. Rasmussen, et al., Panobinostat, a histone deacetylase inhibitor, for latent-virus reactivation in HIV-infected patients on suppressive antiretroviral therapy: a phase 1/2, single group, clinical trial. Lancet HIV 1, e13-21 (2014).
17. O. S. Søgaard, et al., The Depsipeptide Romidepsin Reverses HIV-1 Latency In Vivo. PLoS Pathog. 11, 1-22 (2015).
18. S. A. Migueles, et al., Defective Human Immunodeficiency Virus-Specific CD8+T-Cell Polyfunctionality, Proliferation, and Cytotoxicity Are Not Restored by Antiretroviral Therapy. J. Virol. 83, 11876-89 (2009).
19. M. R. Betts, et al., HIV nonprogressors preferentially maintain highly functional HIV-specific CD8+ T cells. Blood 107, 4781-9 (2006).
20. L. Shan, et al., Stimulation of HIV-1-Specific Cytolytic T Lymphocytes Facilitates Elimination of Latent Viral Reservoir after Virus Reactivation. Immunity 36, 491-501 (2012).
21. K. Deng, et al., Broad CTL response is required to clear latent HIV-1 due to dominance of escape mutations. Nature 517, 381-5 (2015).
22. R. B. Jones, et al., Histone deacetylase inhibitors impair the elimination of hiv-infected cells by cytotoxic T-lymphocytes. PLoS Pathog. 10, e1004287 (2014).
23. R. B. Jones, et al., A Subset of Latency-Reversing Agents Expose HIV-Infected Resting CD4+T-Cells to Recognition by Cytotoxic T-Lymphocytes. PLoS Pathog. 12, 1-25 (2016).
24. V. E. Walker-Sperling, C. W. Pohlmeyer, P. M. Tarwater, J. N. Blankson, The Effect of Latency Reversal Agents on Primary CD8+ T Cells: Implications for Shock and Kill Strategies for Human Immunodeficiency Virus Eradication. EBioMedicine 8, 217-229 (2016).
25. A. K. Kwaa, et al., The effect of ingenol-B on the suppressive capacity of elite suppressor HIV-specific CD8+ T cells. PLoS One 12, e0174516 (2017).
26. S. L. Maude, et al., Chimeric Antigen Receptor T Cells for Sustained Remissions in Leukemia. N. Engl. J. Med. 371, 1507-17 (2014).
27. M. Sadelain, I. Rivière, S. Riddell, Therapeutic T cell engineering. Nat. Publ. Gr. 545, 423-431 (2017).
28. C. H. June, M. Sadelaine, Chimeric Antigen Receptor Therapy. N. Engl. J. Med. 379, 64-73 (2018).
29. M. Subklewe, COUNTERPOINT BiTEs better than CAR T cells. Blood Adv. 5, 607-612 (2021).
30. H. Kantarjian, et al., Blinatumomab versus Chemotherapy for Advanced Acute Lymphoblastic Leukemia. N. Engl. J. Med. 376, 836-847 (2017).
31. T. K. Bera, P. E. Kennedy, E. A. Berger, C. F. Barbas, I. Pastan, Specific killing of HIV-infected lymphocytes by a recombinant immunotoxin directed against the HIV-1 envelope glycoprotein. Mol. Med 4, 384-91 (1998).
32. G. Ferrari, et al., Envelope-specific antibodies and antibody-derived molecules for treating and curing HIV infection. Nat. Rev. Drug Discov. 15, 823-834 (2016).
33. D. D. Sloan, et al., Targeting HIV Reservoir in Infected CD4 T Cells by Dual-Affinity Re-targeting Molecules (DARTs) that Bind HIV Envelope and Recruit Cytotoxic T Cells. PLoS Pathog. 11, e1005233 (2015).
34. J. A. M. Sung, et al., Dual-Affinity Re-Targeting proteins direct T cell-mediated cytolysis of latently HIV-infected cells. J. Clin. Invest. 125, 4077-4090 (2015).
35. C. C. LaBranche, et al., A single amino acid change in the cytoplasmic domain of the simian immunodeficiency virus transmembrane molecule increases envelope glycoprotein expression on infected cells. J. Virol. 69, 5217-27 (1995).
36. R. Byland, P. J. Vance, J. A. Hoxie, M. Marsh, A conserved dileucine motif mediates clathrin and AP-2-dependent endocytosis of the HIV-1 envelope protein. Mol. Biol. Cell 18, 414-25 (2007).
37. M. J. Hogan, et al., Increased surface expression of HIV-1 envelope is associated with improved antibody response in vaccinia prime/protein boost immunization. Virology 514, 106-117 (2018).
38. J. Douglass, et al., Bispecific antibodies targeting mutant RAS neoantigens. Sci. Immunol. 6, eabd5515 (2021).
39. E. H. C. Hsiue, et al., Targeting a neoantigen derived from a common TP53 mutation. Science 371 (2021).
40. P. D. Holler, L. K. Chlewicki, D. M. Kranz, TCRs with high affinity for foreign pMHC show self-reactivity. Nat. Immunol. 4, 55-62 (2003).
41. F. F. González-Galarza, et al., Allele frequency net 2015 update: New features for HLA epitopes, KIR and disease and HLA adverse drug reaction associations. Nucleic Acids Res. 43, D784-8 (2015).
42. M. Andreatta, M. Nielsen, Gapped sequence alignment using artificial neural networks: application to the MHC class I system. Bioinformatics 32, 511-7 (2016).
43. D. B. Keskin, et al., Physical detection of influenza A epitopes identifies a stealth subset on human lung epithelium evading natural CD8 immunity. Proc. Natl. Acad. Sci. 112, 2151-2156 (2015).
44. B. Reinhold, D. B. Keskin, E. L. Reinherz, Molecular detection of targeted major histocompatibility complex I-bound peptides using a probabilistic measure and nanospray MS3 on a hybrid quadrupole-linear ion trap. Anal. Chem. 82, 9090-9099 (2010).
45. B. Reynisson, B. Alvarez, S. Paul, B. Peters, M. Nielsen, NetMHCpan-4.1 and NetMHCIIpan-4.0: improved predictions of MHC antigen presentation by concurrent motif deconvolution and integration of MS MHC eluted ligand data. Nucleic Acids Res. 48, 449-454 (2020).
46. H. Zhang, et al., Novel Single-Cell-Level Phenotypic Assay for Residual Drug Susceptibility and Reduced Replication Capacity of Drug-Resistant Human Immunodeficiency Virus Type 1. J. Virol. 78, 1718-1729 (2004).
47. Y. Sykulev, M. Joo, I. Vturina, T. J. Tsomides, H. N. Eisen, Evidence that a single peptide-MHC complex on a target cell can elicit a cytolytic T cell response. Immunity 4, 565-71 (1996).
48. M. A. Purbhoo, D. J. Irvine, J. B. Huppa, M. M. Davis, T cell killing does not require the formation of a stable mature immunological synapse. Nat. Immunol. 5, 524-30 (2004).
49. R. Dahan, Y. Reiter, T-cell-receptor-like antibodies—generation, function and applications. 14, e6 (2012).
50. A. D. Skora, et al., Generation of MANAbodies specific to HLA-restricted epitopes encoded by somatically mutated genes. Proc. Natl. Acad. Sci. U.S.A. 112, 9967-72 (2015).
51. M. S. Miller, et al., An engineered antibody fragment targeting mutant β-catenin via major histocompatibility complex I neoantigen presentation. J. Biol. Chem. 294, 19322-19334 (2019).
52. R. A. Seder, P. A. Darrah, M. Roederer, T-cell quality in memory and protection: Implications for vaccine design. Nat. Rev. Immunol. 8, 247-58 (2008).
53. M. E. May, et al., Combined Effects of HLA-B*57/5801 Elite Suppressor CD8+ T Cells and NK Cells on HIV-1 Replication. Front. Cell. Infect. Microbiol. 1, 113 (2020).
54. G. B. Cohen, et al., The selective downregulation of class I major histocompatibility complex proteins by HIV-1 protects HIV-infected cells from NK cells. Immunity 10, 661-671 (1999).
55. T. Kerkau, et al., The human immunodeficiency virus type 1 (HIV-1)vpu protein interferes with an early step in the biosynthesis of major histocompatibility complex (MHC) class I molecules. J. Exp. Med. 185, 1295-305 (1997).
56. Y. C. Ho, et al., Replication-competent noninduced proviruses in the latent reservoir increase barrier to HIV-1 cure. Cell 155, 540-551 (2013).
57. O. Lambotte, et al., HIV Controllers: A Homogeneous Group of HIV-1—Infected Patients with Spontaneous Control of Viral Replication. Clin. Infect. Dis. 41, 1053-1056 (2005).
58. S. Grabar, et al., Prevalence and comparative characteristics of long-term nonprogressors and HIV controller patients in the French Hospital Database on HIV. AIDS 23, 1163-9 (2009).
59. K. A. O'Connell, J. R. Bailey, J. N. Blankson, Elucidating the elite: mechanisms of control in HIV-1 infection. Trends Pharmacol. Sci. 30, 631-7 (2009).
60. A. Siez-Cirión, et al., HIV controllers exhibit potent CD8 T cell capacity to suppress HIV infection ex vivo and peculiar cytotoxic T lymphocyte activation phenotype. Proc. Natl. Acad. Sci. U.S.A. 104, 6776-81 (2007).
61. G. D. Gaiha, et al., Structural topology defines protective CD8+ T cell epitopes in the HIV proteome. Science. 364, 480-484 (2019).
62. V. Appay, et al., HIV-specific CD8+ T cells produce antiviral cytokines but are impaired in cytolytic function. J. Exp. Med. 192, 63-75 (2000).
63. S. A. Migueles, et al., Lytic Granule Loading of CD8+ T Cells Is Required for HIV-Infected Cell Elimination Associated with Immune Control. Immunity 29, 1009-21 (2008).
64. G. Bossi, et al., Examining the presentation of tumor-associated antigens on peptide-pulsed T2 cells. Oncoimmunology 2, e26840 (2013).
65. T. J. Tsomides, et al., Naturally processed viral peptides recognized by cytotoxic T lymphocytes on cells chronically infected by human immunodeficiency virus type 1. J. Exp. Med. 180, 1283-93 (1994).
66. F. Kirchhoff, Immune evasion and counteraction of restriction factors by HIV-1 and other primate lentiviruses. Cell Host Microbe 8, 55-67 (2010).
67. Y. Dong, et al., Cryo-EM structures and dynamics of substrate-engaged human 26S proteasome. Nature 565, 49-55 (2019).
68. J. W. Yewdell, E. Reits, J. Neefjes, Making Sense of Mass Destruction: Quantitating MHC Class I Antigen Presentation. Nat. Rev. Immunol. 3, 952-961 (2003).
69. P. Borrow, et al., Antiviral pressure exerted by HIV-1-specific cytotoxic T lymphocytes (CTLs) during primary infection demonstrated by rapid selection of CTL escape virus. Nat. Med. 3, 205-211 (1997).
70. N. Goonetilleke, et al., The first T cell response to transmitted/founder virus contributes to the control of acute viremia in HIV-1 infection. J. Exp. Med. 206, 1253-72 (2009).
71. B. D. Walker, et al., HIV-specific cytotoxic T lymphocytes in seropositive individuals. Nature 328, 345-8 (1987).
72. B. D. Walker, et al., HIV-1 reverse transcriptase is a target for cytotoxic T lymphocytes in infected individuals. Science. 240, 64-6 (1988).
73. M. A. Checkley, B. G. Luttge, E. O. Freed, HIV-1 envelope glycoprotein biosynthesis, trafficking, and incorporation. J. Mol. Biol., 582-608 (2011).
74. W. Fischer, et al., HIV-1 and SARS-CoV-2: Patterns in the Evolution of Two Pandemic Pathogens. Cell Host Microbe 29, 1093-1110 (2021).
75. D. R. Burton, L. Hangartner, Broadly Neutralizing Antibodies to HIV and Their Role in Vaccine Design. Annu. Rev. Immunol 34, 635-59 (2016).
76. J. F. Scheid, et al., HIV-1 antibody 3BNC117 suppresses viral rebound in humans during treatment interruption. Nature 535, 556-560 (2016).
77. S. G. Hansen, et al., Effector memory T cell responses are associated with protection of rhesus monkeys from mucosal simian immunodeficiency virus challenge. Nat. Med. 15, 293-299 (2009).
78. S. G. Hansen, et al., Immune clearance of highly pathogenic SIV infection. Nature 502, 100-104 (2013).
79. S. G. Hansen, et al., Broadly targeted CD8+ T cell responses restricted by major histocompatibility complex E. Science. 351, 714-720 (2016).
80. H. Streeck, et al., Antigen load and viral sequence diversification determine the functional profile of HIV-1-specific CD8+ T cells. PLoS Med. 5, 0790-0803 (2008).
81. D. K. Das, et al., Force-dependent transition in the T-cell receptor β-subunit allosterically regulates peptide discrimination and pMHC bond lifetime. PNAS 112, 1517-22 (2015).
82. Y. Feng, et al., Mechanosensing drives acuity of αβ T-cell recognition. Proc. Natl. Acad. Sci. U.S.A. 114, E8204-E8213 (2017).
83. Y. Feng, E. L. Reinherz, M. J. Lang, αβ T Cell Receptor Mechanosensing Forces out Serial Engagement. Trends Immunol. 39, 596-609 (2018).
84. K. L. Hui, L. Balagopalan, L. E. Samelson, A. Upadhyaya, Cytoskeletal forces during signaling activation in Jurkat T-cells. Mol. Biol. Cell 26, 685-95 (2015).
85. J. Husson, K. Chemin, A. Bohineust, C. Hivroz, N. Henry, Force Generation upon T Cell Receptor Engagement. PLoS One 6, 19680 (2011).
86. S. T. Kim, et al., The as T cell receptor is an anisotropic mechanosensor. J. Biol. Chem. 284, 31028-31037 (2009).
87. Y.-C. Li, et al., Cutting Edge: Mechanical Forces Acting on T cells Immobilized via the TCR Complex Can Trigger TCR Signaling. J Immunol 184, 5959-5963 (2010).
88. B. Liu, W. Chen, B. D. Evavold, C. Zhu, Accumulation of dynamic catch bonds between TCR and agonist peptide-MHC triggers T cell signaling. Cell 157, 357-368 (2014).
89. L. V Sibener, et al., Isolation of a Structural Mechanism for Uncoupling T Cell Receptor Signaling from Peptide-MHC Binding. Cell 174, 672-687 (2018).
90. Rodari A, Darcis G & Van Lint CM (2021) The current status of latency reversing agents for HIV-1 remission. Annu Rev Virol 8(1): 491-514.
91. Bullen C K, Laird G M, Durand C M, Siliciano J D & Siliciano R F (2014) New ex vivo approaches distinguish effective and ineffective single agents for reversing HIV-1 latency in vivo. Nat Med
92. Clayton K L, et al (2021) HIV-infected macrophages resist efficient NK cell-mediated killing while preserving inflammatory cytokine responses. Cell Host Microbe 29(3): 435-447.e9.
93. Y. Fukazawa, et al, B cell follicle sanctuary permits persistent productive simian immunodeficiency virus infection in elite controllers. Nat. Med. 21: 10.1038/nm.3781 [doi]
94. S. H. Huang, et al., (2018) Latent HIV reservoirs exhibit inherent resistance to elimination by CD8+ T cells. J. Clin. Invest, 128 10.1172/JCI97555 [doi].
95. Day C L, et al (2006) PD-1 expression on HIV-specific T cells is associated with T-cell exhaustion and disease progression. Nature 443(7109): 350-354.
96. Borducchi E N, et al (2016) Ad26/MVA therapeutic vaccination with TLR7 stimulation in SIV-infected rhesus monkeys. Nature 540(7632): 284-287.
97. Borducchi E N, et al (2018) Antibody and TLR7 agonist delay viral rebound in SHIV-infected monkeys. Nature 563(7731): 360-364.

1. A T cell receptor (TCR)-mimic (TCRm) antibody comprising two binding domains, wherein (i) a first binding domain specifically binds to a retroviral peptide complexed with a major histocompatibility complex molecule (pMHC) and (ii) a second binding domain specifically binds to T cell signal transduction peptide.

2. The TCRm of claim 1, wherein the retrovirus comprises human immunodeficiency virus (HIV), human T-lymphotropic virus type 1 (HTLV-1) or human T-lymphotropic virus type 2 (HTLV-II).

3. The TCRm of claim 1, wherein the retroviral peptide comprises HIV peptides, HTLV-1 or HTLV-2 peptides.

4. The TCRm of claim 3, wherein the retroviral peptide is an HIV peptide.

5. The TCRm of claim 4, wherein the HIV peptide is an HIV-1 peptide or an HIV-2 peptide.

6. The TCRm of claim 5, wherein the HIV-1 peptide comprises Gag, Pol, Env, or Reverse Transcriptase (RT).

7. The TCRm of claim 1, wherein the major histocompatibility complex (MHC) comprises MHC-I, MHC-II or human leukocyte antigen E (HLA-E).

8. The TCRm of claim 1, wherein the pMHC comprises an HIV-1, Gag, Pol, Env, or Reverse Transcriptase (RT) peptide complexed to MHC-I or HLA-E.

9. The TCRm of claim 1, wherein the T cell signal transduction peptide comprises a CD3 peptide.

10-11. (canceled)

12. The TCRm of claim 1, wherein the antigen binding domains comprise: polyclonal antibodies or fragments thereof, monoclonal antibodies or fragments thereof, antigen-binding antibody fragments, (Fab) fragments, F(ab′)2 fragments, Fab′ fragments, Fv fragments, recombinant IgG (rIgG) fragments, variable heavy chain (VH) regions, single chain antibody fragments, single chain variable fragments (scFv), single domain antibodies, bispecific, antibodies, diabodies, single-chain diabodies (scDb), triabodies, and tetrabodies, tandem di-scFv, or tandem tri-scFvs.

13. The TCRm of claim 12, wherein the antigen binding domains comprise single-chain diabodies (scDb).

14. A method of treating a subject diagnosed with a human immunodeficiency virus (HIV) infection, comprises administering to the subject a therapeutically effective amount of a bispecific T cell receptor (TCR)-mimic (TCRm) antibody comprising two binding domains, wherein (i) a first binding domain specifically binds to an HIV peptide complexed with a major histocompatibility complex molecule (pMHC) and (ii) a second binding domain which specifically binds to T cell signal transduction peptide.

15. The method of claim 14, wherein the HIV peptide is an HIV-1 peptide or an HIV-2 peptide.

16. The method of claim 15, wherein the HIV-1 peptide comprises Gag, Pol, Env, or Reverse Transcriptase (RT).

17. The method of claim 14, wherein the major histocompatibility complex (MHC) comprises MHC-I, MHC-II or human leukocyte antigen E (HLA-E).

18-21. (canceled)

22. The method of claim 14, wherein the antigen binding domains comprise single-chain diabodies (scDb).

23. The method of claim 14, further comprising administering one or more secondary therapeutics.

24. (canceled)

25. A method of producing a bispecific diabodies comprising:
obtaining a biological sample from a subject infected with a virus or infecting a cell in vitro with a virus,
eluting peptides comprising MHC and virus peptides from the biological sample or in vitro infected cells,
utilizing predictive algorithms combined with chromatography and mass spectrometry to identify high-affinity cytotoxic T cell (CTL) epitopes which bind to major histocompatibility complex (MHC) alleles, measuring fragmentation patterns of and elution positions relative to a set of retention time (RT) peptides,
synthesizing identified viral peptides complexed with MHC molecules (pMHC),
screening a phage library to identify single chain variable fragments (scFv) which specifically bind to viral peptides complexed with MHC molecules (pMHC),
converting the scFv fragments to diabodies, and
producing bispecific diabodies.

26-29. (canceled)

30. A method of identifying latency reversing agents (LRAs), comprising:
producing bispecific diabodies of claim 29, which specifically bind to human immunodeficiency virus (HIV) peptides complexed with MHC molecules (pMHC),
screening HIV infected cells against a library of candidate agents;
contacting the HIV infected cells with the pMHC specific diabodies;
assaying for changes in expression or levels of pMHC antigens;
thereby identifying latency reversing agents.

31-34. (canceled)

35. A method of activating an immune response to human immunodeficiency virus (HIV), comprising administering to a subject diagnosed with an HIV infection, a therapeutically effective amount of a T cell receptor (TCR)-mimic (TCRm) antibody comprising two binding domains, wherein (i) a first binding domain specifically binds to an HIV peptide complexed with a major histocompatibility complex molecule (pMHC) and (ii) a second binding domain which specifically binds to T cell signal transduction peptide.

36-39. (canceled)
